(19) United States
(12) Patent Application Publication
(10) Pub. No.: US 2026/0109693 A1
(43) Pub. Date: Apr. 23, 2026

(54) BUTENOLIDE COMPOUNDS CONTAINING THIAZOLIDINONE STRUCTURE AS WELL AS PREPARATION METHOD THEREFOR AND USE THEREOF

(71) Applicant: CHINA AGRICULTURAL UNIVERSITY, Beijing (CN)

(72) Inventors: Mingan WANG, Beijing (CN); Xili LIU, Beijing (CN); Yihao LI, Beijing (CN); Tingting ZHANG, Beijing (CN); Qian ZHANG, Beijing (CN)

(21) Appl. No.: 19/114,226

(22) PCT Filed: Oct. 27, 2023
(86) PCT No.: PCT/CN2023/127209

§ 371 Date: Mar. 21, 2025

(30) Foreign Application Priority Data
Mar. 31, 2023 (CN) 202310338419.7

(51) Int. Cl. C07D 417/06; A01N 43/78; A01P 3/00

(52) U.S. Cl. CPC C07D 417/06; A01N 43/78; A01P 3/00

(57) ABSTRACT

The present application relates to the technical field of agricultural chemistry, in particular to butenolide compounds containing a thiazolidinone structure as well as a preparation method therefor and the use thereof. The butenolide compounds containing a thiazolidinone structure are characterized by having the following structural general formula (I). The compounds disclosed by the present application have excellent fungicidal activity on various pathogens in agriculture or other fields, and can favorably protect important crops and domestic animals in agriculture and horticulture industry, and the environment that the human beings rely on from invasion of pathogens.

### CROSS REFERENCE

The present application claims priority to Chinese Patent Application No. 2023103384197, entitled “BUTENOLIDE COMPOUNDS CONTAINING THIAZOLIDINONE STRUCTURE AS WELL AS PREPARATION METHOD THEREFOR AND USE THEREOF”, filed on Mar. 31, 2023, the entire disclosure of which is incorporated herein by reference.

### TECHNICAL FIELD

The present application relates to the field of agricultural chemistry technology, particularly to a butenolide compound containing a thiazolidinone structure as well as a preparation method therefor and the use thereof.

### BACKGROUND ART

Spirocyclic butenolide compounds are widely present in nature, such as spirofragilid, lambertellol A, crossalactone D, and pyrenolide D. These natural products have various excellent biological activities such as insecticidal, fungicidal, spore germination inhibition, anti-inflammatory, and anti-tumor activities. Commercially available spirocyclic butenolide pesticide varieties include spirodiclofen, spiromesifen, spirotetramat, and spiropidion, which are a type of excellent agricultural insecticides and acaricides. It can be seen that spirocyclic butenolide is an excellent pharmacophore worthy of further research and development.

For example, patent CN111574507A discloses a compound containing a natural butenolide skeleton, preparation and use thereof, which has good control effects on various pests and plant pathogenic fungi. Patent CN110396083A discloses pyridazinone group-containing butenolide compounds, which have excellent control effects on common diseases of various important crops in agriculture and horticulture. The above-mentioned existing technologies have proven that butenolide compounds have excellent effects on controlling plant diseases and the like. However, the above-mentioned patents have shortcomings such as insufficient stability of the compounds or long synthesis schemes, and high costs. In addition, patent CN104370891 discloses that 5-(butenolide-3-ethylidene)-2-aminoimidazolinone compounds have good fungicidal effects on various plant pathogens such as rice sheath blight, sclerotinia rot of colza, and pepper phytophthora. However, the toxicity test of the above compounds shows that their toxicities were relatively low, there is still a certain gap from commercial practical application, and there is still a lot of room for improvement in their fungistatic activity.

It can be seen that there is still room for further improvement in the use of butenolide compounds for the prevention and control of pests and diseases in the field of agricultural chemistry.

### SUMMARY OF THE APPLICATION

The purpose of the present application is to provide a butenolide compound containing a thiazolidinone structure that can prevent and control various plant pathogens, as well as preparation method therefor and use thereof.

To achieve the above purpose, the technical solution of the present application is as follows:

The present application first provides a butenolide compound containing a thiazolidinone structure, having the following general formula:

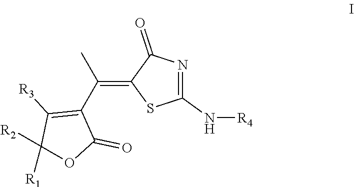

in the formula:
R₁ and R₂ are each independently selected from hydrogen and C₁-C₁₂ alkyl; or, R₁ and R₂, together with the carbon atom bonded therewith, form C₃-C₁₂ cycloalkyl or C₃-C₁₂ cyclic heteroalkyl, heteroatom in the heteroalkyl is N, O, or S, wherein the hydrogen in the C₃-C₁₂ cycloalkyl or the C₃-C₁₂ cyclic heteroalkyl may be monosubstituted or polysubstituted by R₅;
R₃ is selected from hydrogen, hydroxyl, C₁-C₁₂ alkyl, C₅-C₇ cycloalkyl, unsubstituted aryl or heteroaryl or aryl or heteroaryl containing one to three R₆ substituents:
R₄ is selected from C₁-C₁₂ alkyl, halogenated C₁-C₁₂ alkyl, C₁-C₁₂ alkoxy, halogenated C₁-C₁₂ alkoxy, C₃-C₁₂ cycloalkyl, aryl or heteroaryl substituted by one to three R₆, or biphenyl- or phenoxyphenyl substituted by one or more R₆;
R₅ is selected from hydroxyl, carbonyl, methoxy, methoxyoximido, formyl, halogen, C₁-C₁₂ alkyl, halogenated C₁-C₁₂ alkyl, C₁-C₁₂ alkoxy, or halogenated C₁-C₁₂ alkoxy; and
R₆ is selected from halogen, hydroxyl, amino, cyano, nitro, C₁-C₁₂ alkyl, halogenated C₁-C₁₂ alkyl, C₁-C₁₂ alkoxy, halogenated C₁-C₁₂ alkoxy, C₃-C₁₂ cycloalkyl, C₁-C₁₂ alkylthio, halogenated C₁-C₁₂ alkylthio, C₁-C₁₂ alkylamino, halogenated C₁-C₁₂ alkylamino, di(C₁-C₁₂ alkyl)amino, halogenated di(C₁-C₁₂ alkyl)amino, C₂-C₁₂ alkenyl, C₂-C₁₂ alkynyl, C₂-C₁₂ alkenoxy, halogenated C₂-C₁₂ alkenoxy, C₂-C₁₂ alkynyloxy, halogenated C₂-C₁₂ alkynyloxy, C₁-C₁₂ alkylsulfonyl, halogenated C₁-C₁₂ alkylsulfonyl, C₁-C₁₂ alkylcarbonyl, halogenated C₁-C₁₂ alkylcarbonyl, C₁-C₁₂ alkoxy carbonyl, halogenated C₁-C₁₂ alkoxycarbonyl, C₁-C₁₂ alkoxy C₁-C₁₂ alkyl, halogenated C₁-C₁₂ alkoxy C₁-C₁₂ alkyl, C₁-C₁₂ alkylthio C₁-C₁₂ alkyl, halogenated C₁-C₁₂ alkylthio C₁-C₁₂ alkyl, C₁-C₁₂ alkoxycarbonyl C₁-C₁₂ alkyl, halogenated C₁-C₁₂ alkoxycarbonyl C₁-C₁₂ alkyl, C₁-C₁₂ alkylthiocarbonyl C₁-C₁₂ alkyl, halogenated C₁-C₁₂ alkylthiocarbonyl C₁-C₁₂ alkyl, C₁-C₁₂ alkylcarbonyloxy, halogenated C₁-C₁₂ alkylcarbonyloxy, C₁-C₁₂ alkoxycarbonyloxy, halogenated C₁-C₁₂ alkoxycarbonyloxy, C₁-C₁₂ alkylsulfonyloxy, halogenated C₁-C₁₂ alkylsulfonyloxy, C₁-C₁₂ alkoxy C₁-C₁₂ alkoxy or halogenated C₁-C₁₂ alkoxy C₁-C₁₂ alkoxy.

The present application has found that introduction of thiazolidinone into butenolide compound can significantly enhance the fungistatic activity of butenolide compound, greatly improve their toxicity, and significantly enhance their fungistatic effect.

The terms used in the definition of compound of general formula I given above are generally defined as follows:

Halogen: referring to fluorine, chlorine, bromine, or iodine.

Alkyl: a linear or branched alkyl, such as methyl, ethyl, propyl, isopropyl, n-butyl, or tert-butyl.

Cycloalkyl: a substituted or unsubstituted cyclic alkyl, such as cyclopropyl, cyclopentyl, cyclohexyl, or substituted cyclopropyl, cyclopentyl, and cyclohexyl, with substituents such as alkyl, and halogen.

Halogenated alkyl: a linear or branched alkyl in which the hydrogen atoms on these alkyls can be partially or completely replaced by halogen atoms, such as chloromethyl, dichloromethyl, trichloromethyl, fluoromethyl, difluoromethyl, and trifluoromethyl.

Alkylsulfonyl: a linear or branched alkyl connected to a structure via a sulfinyl group (—SO₂), such as methylsulfonyl.

Halogenated alkylsulfonyl: a linear or branched alkylsulfonyl, in which the hydrogen atoms on the alkyl can be partially or completely replaced by halogen atoms.

Alkylaminothio: such as CH₃NHS—, and C₂H₅NHS—.

Dialkylaminothio: such as (CH₃)₂NS—, and (C₂H₅)₂NS—.

Alkylaminosulfonyl: such as alkyl-NH—SO₂—.

Dialkylaminosulfonyl: such as (alkyl)₂-N—SO₂—.

Alkylsulfonylaminocarbonyl: such as alkyl-SO₂—NH—CO—.

Alkylcarbonylaminosulfonyl: such as alkyl-CO—NH—SO₂—.

Alkylcarbonylalkyl: such as alkyl-CO-alkyl-.

Alkylsulfonyloxy: such as alkyl-S(O)₂—O—.

Halogenated alkylsulfonyloxy: the hydrogen atoms on the alkyl of alkylsulfonyloxy can be partially or completely replaced by halogen atoms, such as CF₃—SO₂—O.

Cycloalkoxycarbonyl: such as cyclopropoxycarbonyl, and cyclohexyloxycarbonyl.

Alkoxy: a linear or branched alkyl that is connected to a structure via an oxygen atom bond.

Halogenated alkoxy: a linear or branched alkoxy, the hydrogen atoms on these alkoxys can be partially or completely replaced by halogen atoms, such as chloromethoxy, dichloromethoxy, trichloromethoxy, fluoromethoxy, difluoromethoxy, trifluoromethoxy, chlorofluoromethoxy, and trifluoroethoxy.

Halogenated alkoxycarbonyl: the hydrogen atoms on the alkyl of alkoxycarbonyl can be partially or completely replaced by halogen atoms, such as ClCH₂CH₂OCO—, and CF₃CH₂OCO—.

Alkoxyalkyl: alkyl-O-alkyl-, such as CH₃OCH₂—.

Halogenated alkoxyalkyl: the hydrogen atoms on the alkyl of alkoxyalkyl can be partially or completely replaced by halogen atoms, such as ClCH₂CH₂OCH₂—, and CF₃CH₂OCH₂—.

Alkoxycarbonylalkyl: alkoxycarbonyl-alkyl-, such as CH₃OCOCH₂—.

Halogenated alkoxycarbonylalkyl: the hydrogen atoms on the alkyl of alkoxycarbonylalkyl are partially or completely replaced by halogen atoms, such as CF₃CH₂OCOCH₂—.

Alkylcarbonyloxy: such as CH₃COO—.

Halogenated alkylcarbonyloxy: the hydrogen atoms of alkylcarbonyloxy can be partially or completely replaced by halogen atoms, such as CF₃COO—.

Alkoxycarbonyloxy: alkoxycarbonyl-oxy-, such as CH₃OCOO—.

Halogenated alkoxycarbonyloxy: the hydrogen atoms on the alkyl of alkoxycarbonyloxy can be partially or completely replaced by halogen atoms, such as CF₃OCOO—.

Alkylthiocarbonylalkyl: alkylthiocarbonyl-alkyl-, such as CH₃SCOCH₂—.

Halogenated alkylthiocarbonylalkyl: the hydrogen atoms on the alkyl of alkylthiocarbonylalkyl can be partially or completely replaced by halogen atoms, such as CF₃CH₂SCOCH₂—.

Alkoxyalkoxy: such as CH₃OCH₂O—.

Halogenated alkoxyalkoxy: the hydrogen atoms on the alkoxy can be partially or completely replaced by halogen atoms, such as CF₃OCH₂O—.

Alkoxyalkoxycarbonyl: such as CH₃OCH₂CH₂OCO—.

Alkylthio: a linear or branched alkyl connected to a structure via a sulfur atom bond.

Halogenated alkylthio: a linear or branched alkylthio, in which the hydrogen atoms on these alkyls can be partially or completely replaced by halogen atoms, such as chloromethylthio, dichloromethylthio, trichloromethylthio, trifluoromethylthio, and chlorofluoromethylthio.

Alkylthioalkyl: alkyl-S-alkyl-, such as CH₃SCH₂—.

Halogenated alkylthioalkyl: the hydrogen atoms on the alkyl of alkylthioalkyl can be partially or completely replaced by halogen atoms, such as ClCH₂CH₂SCH₂—, and CF₃CH₂SCH₂—.

Alkylamino: a linear or branched alkyl connected to a structure via a nitrogen atom bond.

Halogenated alkylamino: a linear or branched alkylamino, the hydrogen atoms on these alkyls can be partially or completely replaced by halogen atoms.

Dialkylamino: such as (CH₃)₂N—, and (CH₃CH₂)₂N—.

Halogenated dialkylamino: the hydrogen atoms on the alkyl can be partially or completely replaced by halogen atoms, such as (CF₃)₂N—, and (CF₃CH₂)₂N—.

Alkenyl: linear or branched alkenes, such as ethenyl, 1-propenyl, 2-propenyl, and different butenyl, pentenyl, and hexenyl isomers. Alkenyl also includes polyenes, such as 1,2-propadienyl and 2,4-hexadienyl.

Halogenated alkenyl: linear or branched alkenes, the hydrogen atoms on these alkenyls can be partially or completely replaced by halogen.

Alkenoxy: linear or branched alkenes that is connected to a structure via an oxygen atom bond.

Halogenated alkenoxy: a linear or branched alkenoxy, the hydrogen atoms on these alkenoxys can be partially or completely replaced by halogen atoms.

Alkynyl: linear or branched alkynes, such as acetenyl, 1-propynyl, 2-propynyl, and different butynyl, pentynyl, and hexynyl isomers. Alkynyl also includes functional groups composed of multiple triple bonds, such as 2,5-hexadiynyl.

Halogenated alkynyl: linear or branched alkynes, the hydrogen atoms on these alkynyls can be partially or completely replaced by halogen atoms.

Alkylcarbonyl: the alkyl is connected to a structure via a carbonyl, such as CH₃CO—, and CH₃CH₂CO—.

Halogenated alkylcarbonyl: the hydrogen atoms on the alkyl of alkylcarbonyl can be partially or completely replaced by halogen atoms, such as CF₃CO—.

Alkoxycarbonyl: the alkoxy is connected to a structure via a carbonyl, such as CH₃OCO—, and CH₃CH₂OCO—.

Aminocarbonyl: such as NH₂CO—.

Alkylaminocarbonyl: alkyl-NH—CO—, such as CH₃NHCO—.

Dialkylaminocarbonyl: such as (CH₃)₂NCO—, and (CH₃CH₂)₂NCO—. The aryl portion of (hetero)aryl, (hetero)arylalkyl, (hetero)arylcarbonyl, (hetero)arylmethylcarbonyl, (hetero)arylcarbonylalkyl, (hetero)aryloxycarbonyl, and (hetero)arylalkyloxycarbonyl includes phenyl or naphthyl.

Heteroaryl is a five membered or six-membered ring containing one or more of N, O, and S heteroatoms, such as furanyl, pyrazolyl, thiazolyl, pyridinyl, pyrimidinyl, pyrazinyl, pyridazinyl, triazinyl, and quinolinyl.

(Hetero)aryl: such as phenyl.

(Hetero)arylalkyl: such as benzyl, phenethyl, and p-fluorobenzyl.

(Hetero)arylcarbonyl: such as benzoyl, and 4-chlorobenzoyl.

(Hetero)arylmethylcarbonyl: such as PhCH₂CO—. (Hetero)arylcarbonylalkyl: such as PhCOCH₂—.

(Hetero)aryloxycarbonyl: such as phenoxycarbonyl, 4-fluorophenoxycarbonyl, 4-chlorophenoxycarbonyl, and naphthoxycarbonyl.

Arylalkyloxycarbonyl: such as benzyloxycarbonyl, 4-chlorobenzyloxycarbonyl, and 4-trifluoromethylbenzyloxycarbonyl.

(Hetero)arylalkyloxycarbonyl: such as PhCH₂OCO—, and 4-ClPhCH₂OCO.

Preferably, in the compounds represented by the general formula I:
R₁ and R₂ are each independently selected from hydrogen or C₁-C₄ alkyl: or, R₁ and R₂, together with the carbon atom bonded therewith, can form C₅-C₆ cycloalkyl or C₅-C₆ cyclic heteroalkyl, the heteroatom in the heteroalkyl being N, O, or S, wherein the hydrogen in the C₃-C₁₂ cycloalkyl or the C₃-C₁₂ cyclic heteroalkyl can be monosubstituted by R₅;
R₃ is selected from hydrogen, C₁-C₄ alkyl, C₁-C₄ cycloalkyl, unsubstituted aryl or heteroaryl or aryl or heteroaryl containing one to three R₆ substituents:
R₄ is selected from aryl or heteroaryl substituted by one to three R₆, biphenyl- or phenoxyphenyl substituted by one or two R₆;
R₅ is selected from hydroxyl, carbonyl, methoxy, methoxyoximido, formyl, halogen, C₁-C₄ alkyl, and halogenated C₁-C₄ alkyl:
R₆ is selected from halogen, hydroxyl, amino, cyano, nitro, C₁-C₄ alkyl, halogenated C₁-C₄ alkyl, C₁-C₄ alkoxy, halogenated C₁-C₄ alkoxy, C₃-C₄ cycloalkyl, C₁-C₄ alkylthio, halogenated C₁-C₄ alkylthio, C₁-C₄ alkylamino, halogenated C₁-C₄ alkylamino, C₁-C₄ alkylsulfonyl, halogenated C₁-C₄ alkylsulfonyl, C₁-C₄ alkylcarbonyl, and halogenated C₁-C₄ alkylcarbonyl.

Preferably, in the compounds represented by the general formula I:
R₁ and R₂ are each independently selected from hydrogen, methyl, ethyl, n-propyl, isopropyl, n-butyl, sec-butyl, isobutyl, tert-butyl, cyclopropyl, or cyclobutyl: or, R₁ and R₂, together with the carbon atom bonded therewith, can form a five membered or six-membered carbon ring, and the hydrogen on the carbon ring can be monosubstituted by R₅:
R₃ is selected from hydrogen, methyl, ethyl, n-propyl, isopropyl, n-butyl, sec-butyl, isobutyl, tert-butyl, cyclopropyl, cyclobutyl, unsubstituted aryl or heteroaryl or aryl or heteroaryl substituted by one to three R₆:
R₄ is selected from aryl or heteroaryl substituted by one to three R₆, and biphenyl- or phenoxyphenyl substituted by one or two R₆;
R₅ is selected from hydroxyl, carbonyl, methoxy, methoxyoximido, or halogen;
R₆ is selected from halogen, hydroxyl, amino, cyano, nitro, C₁-C₄ alkyl, halogenated C₁-C₄ alkyl, C₁-C₄ alkoxy, halogenated C₁-C₄ alkoxy, C₁-C₄ alkylthio or halogenated C₁-C₄ alkylthio.

Preferably, in the compounds represented by the general formula I:
R₁ and R₂ are methyl respectively:
R₁ and R₂, together with the carbon atom bonded therewith, can form a saturated five membered or six-membered carbon ring, and the hydrogen on the carbon ring can be monosubstituted by R₅:
R₃ is selected from methyl or phenyl substituted by R₆;
R₄ is selected from phenyl substituted by R₆, biphenyl- or phenoxyphenyl substituted by one or two R₆:
R₅ is selected from methoxy or methoxyoximido:
R₆ is selected from methyl, tert-butyl, halogen, nitro, methylthio, methoxy, or trifluoromethyl.

The preferred compounds represented by the general formula I in the present application include the following compounds:

When R₁=R₂=—(CH₂)₅—, R₃=CH₃, as shown in table 1, the substituent R₄ corresponds sequentially to 1-21 in table 1, the representative compound numbers are I-1-1 to I-1-21.

When R₁=R₂=CH₃, R₃=CH₃, as shown in table 1, the substituent R₄ corresponds sequentially to 1-21 in table 1, the representative compound numbers are I-2-1 to I-2-21.

When R₁=R₂=CH₃, R₃=Ph, as shown in table 1, the substituent R₄ corresponds to 1-21 in table 1, the representative compound numbers are I-3-1 to I-3-21.

When R₁-R₂=—(CH₂)₅—, R₃=Ph, as shown in table 1, the substituent R₄ corresponds sequentially to 1-21 in table 1, the representative compound numbers are I-4-1 to I-4-21.

When R₁=R₂=—(CH₂)₅—, R₃=4-FPh, as shown in table 1, the substituent R₄ corresponds sequentially to 1-21 in table 1, the representative compound numbers are I-5-1 to I-5-21.

When R₁=R₂=—(CH₂)₅—, R₃=4-ClPh, as shown in table 1, the substituent R₄ corresponds sequentially to 1-21 in table 1, the representative compound numbers are I-6-1 to I-6-21.

When R₁=R₂=—(CH₂)₅—, R₃=4-BrPh, as shown in table 1, the substituent R₄ corresponds sequentially to 1-21 in table 1, the representative compound numbers are I-7-1 to I-7-21.

When R₁=R₂=—(CH₂)₅—, R₃=4-C₄H₉Ph, as shown in table 1, the substituent R₄ corresponds sequentially to 1-21 in table 1, the representative compound numbers are I-8-1 to I-8-21.

When R₁=R₂=—(CH₂)₅—, R₃=4-OCH₃Ph, as shown in table 1, the substituent R₄ corresponds sequentially to 1-21 in table 1, the representative compound numbers are I-9-1 to I-9-21.

When R₁=R₂=—(CH₂)₅—, R₃=4-SCH₃Ph, as shown in table 1, the substituent R₄ corresponds sequentially to 1-21 in table 1, the representative compound numbers are I-10-1 to I-10-21.

When R₁=R₂=—(CH₂)₅—, R₃=4-SO₂Ph, as shown in table 1, the substituent R₄ corresponds sequentially to 1-21 in table 1, the representative compound numbers are I-11-1 to I-11-21.

When R₁=R₂=—(CH₂)₅—, R₃=4-CF₃Ph, as shown in table 1, the substituent R₄ corresponds sequentially to 1-21 in table 1, the representative compound numbers are I-12-1 to I-12-21.

When R₁=R₂=—(CH₂)₅—, R₃₌₃-BrPh, as shown in table 1, the substituent R₄ corresponds sequentially to 1-21 in table 1, the representative compound numbers are I-13-1 to I-13-21.

When R₁=R₂=—(CH₂)₅—, R₃₌₃-ClPh, as shown in table 1, the substituent R₄ corresponds sequentially to 1-21 in table 1, the representative compound numbers are I-14-1 to I-14-21.

When R₁=R₂=—(CH₂)₅—, R₃₌₃-FPh, as shown in table 1, the substituent R₄ corresponds sequentially to 1-21 in table 1, the representative compound numbers are I-15-1 to I-15-21.

When R₁=R₂=—(CH₂)₅—, R₃₌₃-CF₃Ph, as shown in table 1, the substituent R₄ corresponds sequentially to 1-21 in table 1, the representative compound numbers are I-16-1 to I-16-21.

When R₁=R₂=—(CH₂)₅—, R₃₌₃-NO2Ph, as shown in table 1, the substituent R₄ corresponds sequentially to 1-21 in table 1, the representative compound numbers are I-17-1 to I-17-21.

When R₁=R₂=—(CH₂)₅—, R₃₌₃-OCH₃Ph, as shown in table 1, the substituent R₄ corresponds sequentially to 1-21 in table 1, the representative compound numbers are I-18-1 to I-18-21.

When R₁=R₂=—(CH₂)₅—, R₃₌₂-BrPh, as shown in table 1, the substituent R₄ corresponds sequentially to 1-21 in table 1, the representative compound numbers are I-19-1 to I-19-21. When R₁=R₂=—(CH₂)₅—, R₃₌₂-ClPh, as shown in table 1, the substituent R₄ corresponds sequentially to 1-21 in table 1, the representative compound numbers are I-20-1 to I-20-21. When R₁=R₂=—(CH₂)₅—, R₃₌₂-FPh, as shown in table 1, the substituent R₄ corresponds sequentially to 1-21 in table 1, the representative compound numbers are I-21-1 to I-21-21.

When R₁=R₂=—CH₂CH₂CHOCH₃CH₂CH₂—, R₃=CH₃, as shown in table 1, the substituent R₄ corresponds sequentially to 1-21 in table 1, the representative compound numbers are I-22-1 to I-22-21.

When R₁=R₂=—CH₂CH₂CHOCH₃CH₂CH₂—, R₃=Ph, as shown in table 1, the substituent R₄ corresponds sequentially to 1-21 in table 1, the representative compound numbers are I-23-1 to I-23-21.

When R₁=R₂=—CH₂CH₂CNOCH₃CH₂CH₂—, R₃=CH₃, as shown in table 1, the substituent R₄ corresponds sequentially to 1-21 in table 1, the representative compound numbers are I-24-1 to I-24-21.

When R₁=R₂=—CH₂CH₂CNOCH₃CH₂CH₂—, R₃=Ph, as shown in table 1, the substituent R₄ corresponds sequentially to 1-21 in table 1, the representative compound numbers are I-25-1 to I-25-21.

When R₁=R₂=—CH₂CH₂CHOHCH₂CH₂—, R₃=CH₃, as shown in table 1, the substituent R₄ corresponds sequentially to 1-21 in table 1, the representative compound numbers are I-26-1 to I-26-21.

When R₁=R₂=—CH₂CH₂CHOHCH₂CH₂—, R₃=Ph, as shown in table 1, the substituent R₄ corresponds sequentially to 1-21 in table 1, the representative compound numbers are I-27-1 to I-27-21.

When R₁=R₂=—CH₂CH₂COCH₂CH₂—, R₃=CH₃, as shown in table 1, the substituent R₄ corresponds sequentially to 1-21 in table 1, the representative compound numbers are I-28-1 to I-28-21.

When R₁=R₂=—CH₂CH₂COCH₂CH₂—, R₃=Ph, as shown in table 1, the substituent R₄ corresponds sequentially to 1-21 in table 1, the representative compound numbers are I-29-1 to I-29-21.

When R₁=R₂=—CH₂CH₂CHFCH₂CH₂—, R₃=CH₃, as shown in table 1, the substituent R₄ corresponds sequentially to 1-21 in table 1, the representative compound numbers are I-30-1 to I-30-21.

When R₁=R₂=—CH₂CH₂CHFCH₂CH₂—, R₃=Ph, as shown in table 1, the substituent R₄ corresponds sequentially to 1-21 in table 1, the representative compound numbers are I-31-1 to I-31-21.

When R₁=R₂=—CH₂CH₂CHClCH₂CH₂—, R₃=CH₃, as shown in table 1, the substituent R₄ corresponds sequentially to 1-21 in table 1, the representative compound numbers are I-32-1 to I-32-21.

When R₁=R₂=—CH₂CH₂CHClCH₂CH₂—, R₃=Ph, as shown in table 1, the substituent R₄ corresponds sequentially to 1-21 in table 1, the representative compound numbers are I-33-1 to I-33-21.

When R₁=R₂=—CH₂CH₂CHBrCH₂CH₂—, R₃=CH₃, as shown in table 1, the substituent R₄ corresponds sequentially to 1-21 in table 1, the representative compound numbers are I-34-1 to I-34-21.

When R₁=R₂=—CH₂CH₂CHBrCH₂CH₂—, R₃=Ph, as shown in table 1, the substituent R₄ corresponds sequentially to 1-21 in table 1, the representative compound numbers are I-35-1 to I-35-21.

When R₁=R₂=CH₃, R₃=CH₃NH, as shown in table 1, the substituent R₄ corresponds sequentially to 1-21 in table 1, the representative compound numbers are I-36-1 to I-36-21.

When R₁=R₂=—(CH₂)₅—, R₃=CH₃NH, as shown in table 1, the substituent R₄ corresponds sequentially to 1-21 in table 1, the representative compound numbers are I-37-1 to I-37-21. When R₁=R₂=CH₃, R₃=(CH₃)₂N, as shown in table 1, the substituent R₄ corresponds sequentially to 1-21 in table 1, the representative compound numbers are I-38-1 to I-38-21. When R₁=R₂=—(CH₂)₅—, R₃=(CH₃)₂N, as shown in table 1, the substituent R₄ corresponds sequentially to 1-21 in table 1, the representative compound numbers are I-39-1 to I-39-21.

When R₁=R₂=CH₃, R₃-C₁, as shown in table 1, the substituent R₄ corresponds sequentially to 1-21 in table 1, the representative compound numbers are I-40-1 to I-40-21.

When R₁=R₂=—(CH₂)₅—, R₃-C₁, as shown in table 1, the substituent R₄ corresponds sequentially to 1-21 in table 1, the representative compound numbers are I-41-1 to I-41-21.

When R₁=R₂=CH₃, R₃=F, as shown in table 1, the substituent R₄ corresponds sequentially to 1-21 in table 1, the representative compound numbers are I-42-1 to I-42-21.

When R₁=R₂=—(CH₂)₅—, R₃=F, as shown in table 1, the substituent R₄ corresponds sequentially to 1-21 in table 1, the representative compound numbers are I-43-1 to I-43-21. When R₁=R₂=CH₃, R₃=Br, as shown in table 1, the substituent R₄ corresponds sequentially to 1-21 in table 1, the representative compound numbers are I-44-1 to I-44-21.

When R₁=R₂=—(CH₂)₅—, R₃=Br, as shown in table 1, the substituent R₄ corresponds sequentially to 1-21 in table 1, the representative compound numbers are I-45-1 to I-45-21.

When R₁=R₂=—(CH₂)₅—, R₃=C₅H₄N, as shown in table 1, the substituent R₄ corresponds sequentially to 1-21 in table 1, the representative compound numbers are I-46-1 to I-46-21.

When R₁=R₂=CH₃, R₃=C₅H₄N, as shown in table 1, the substituent R₄ corresponds sequentially to 1-21 in table 1, the representative compound numbers are I-47-1 to I-47-21.

Among them, in the above n-R₃Ph, n represents the substitution position of R₃ on the benzene ring, for example, 4-FPh refers to 4-fluorophenyl, and 3-OCH₃Ph refers to 3-methoxyphenyl.

TABLE 1

| No. | R₄ | No. | R₄ | No. | R₄ |
|---|---|---|---|---|---|
| 1 | H | 2 | CH₃ | 3 | C₂H₅ |
| 4 | n-C₃H₇ | 5 | n-C₄H₉ | 6 | i-C₄H₉ |
| 7 | 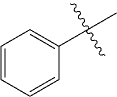 [SMILES: *c1ccccc1] | 8 | 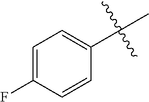 [SMILES: *c1ccc(F)cc1] | 9 | 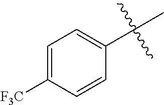 [SMILES: *c1ccc(C(F)(F)F)cc1] |
| 10 | 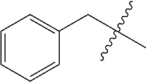 [SMILES: *Cc1ccccc1] | 11 | 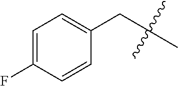 [SMILES: *Cc1ccc(F)cc1] | 12 | 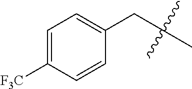 [SMILES: *Cc1ccc(C(F)(F)F)cc1] |
| 13 | 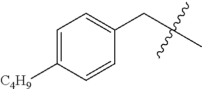 | 14 | 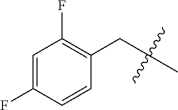 [SMILES: *Cc1ccc(F)cc1F] | 15 | 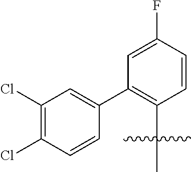 [SMILES: *c1ccc(F)cc1-c1ccc(Cl)c(Cl)c1] |
| 16 | 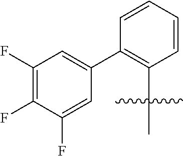 [SMILES: *c1ccccc1-c1cc(F)c(F)c(F)c1] | 17 | 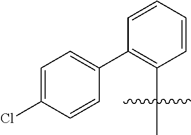 [SMILES: *c1ccccc1-c1ccc(Cl)cc1] | 18 | 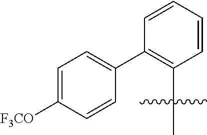 [SMILES: *c1ccccc1-c1ccc(OC(F)(F)F)cc1] |
| 19 | 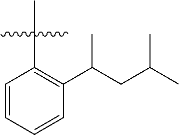 [SMILES: *c1ccccc1C(C)CC(C)C] | 20 | 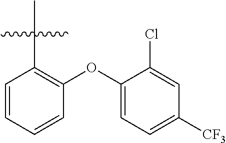 [SMILES: *c1ccccc1Oc1ccc(C(F)(F)F)cc1Cl] | 21 | 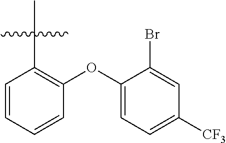 [SMILES: *c1ccccc1Oc1ccc(C(F)(F)F)cc1Br] |

The present application also provides an intermediate of butenolide compounds containing a thiazolidinone structure, the intermediate has a structure represented by the general formula 3:

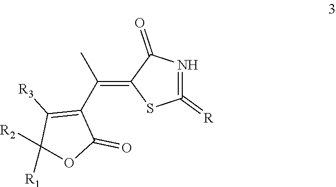

R is one selected from NH and S;
R₁ and R₂ are each independently selected from hydrogen or C₁-C₁₂ alkyl; or, R₁ and R₂, together with the carbon atom bonded therewith, form C₃-C₁₂ cycloalkyl or C₃-C₁₂ cyclic heteroalkyl, the heteroatom in the heteroalkyl being N, O, or S, wherein the hydrogen in the C₃-C₁₂ cycloalkyl or the C₃-C₁₂ cyclic heteroalkyl may be monosubstituted or polysubstituted by R₅:
R₃ is selected from hydrogen, hydroxyl, C₁-C₁₂ alkyl, C₅-C₇ cycloalkyl, unsubstituted aryl or heteroaryl or aryl or heteroaryl containing 1-3 R₆ substituents:
R₅ is selected from hydroxyl, carbonyl, methoxy, methoxyoximido, formyl, halogen, C₁-C₁₂ alkyl, halogenated C₁-C₁₂ alkyl, C₁-C₁₂ alkoxy, or halogenated C₁-C₁₂ alkoxy; and
R₆ is selected from halogen, hydroxyl, amino, cyano, nitro, C₁-C₁₂ alkyl, halogenated C₁-C₁₂ alkyl, C₁-C₁₂ alkoxy, halogenated C₁-C₁₂ alkoxy, C₃-C₁₂ cycloalkyl, C₁-C₁₂ alkylthio, halogenated C₁-C₁₂ alkylthio, C₁-C₁₂ alkylamino, halogenated C₁-C₁₂ alkylamino, di(C₁-C₁₂ alkyl)amino, halogenated di(C₁-C₁₂ alkyl)amino, C₂-C₁₂ alkenyl, C₂-C₁₂ alkynyl, C₂-C₁₂ alkenoxy, halogenated C₂-C₁₂ alkenoxy, C₂-C₁₂ alkynyloxy, halogenated C₂-C₁₂ alkynyloxy, C₁-C₁₂ alkylsulfonyl, halogenated C₁-C₁₂ alkylsulfonyl, C₁-C₁₂ alkylcarbonyl, halogenated C₁-C₁₂ alkylcarbonyl, C₁-C₁₂ alkoxycarbonyl, halogenated C₁-C₁₂ alkoxycarbonyl, C₁-C₁₂ alkoxy C₁-C₁₂ alkyl, halogenated C₁-C₁₂ alkoxy C₁-C₁₂ alkyl, C₁-C₁₂ alkylthio C₁-C₁₂ alkyl, halogenated C₁-C₁₂ alkylthio C₁-C₁₂ alkyl, C₁-C₁₂ alkoxycarbonyl C₁-C₁₂ alkyl, halogenated C₁-C₁₂ alkoxycarbonyl C₁-C₁₂ alkyl, C₁-C₁₂ alkylthiocarbonyl C₁-C₁₂ alkyl, halogenated C₁-C₁₂ alkylthiocarbonyl C₁-C₁₂ alkyl, C₁-C₁₂ alkylcarbonyloxy, halogenated C₁-C₁₂ alkylcarbonyloxy, C₁-C₁₂ alkoxycarbonyloxy, halogenated C₁-C₁₂ alkoxycarbonyloxy, C₁-C₁₂ alkylsulfonyloxy, halogenated C₁-C₁₂ alkylsulfonyloxy, C₁-C₁₂ alkoxy C₁-C₁₂ alkoxy or halogenated C₁-C₁₂ alkoxy C₁-C₁₂ alkoxy.

During the optimization of the intermediates in the present application, it was unexpectedly discovered that in the intermediate compounds with the structure represented by the general formula 3, when R is one selected from NH and S, the compounds have good fungistatic activity similar to the compounds represented by the general formula I, and the fungistatic effect is very outstanding. When R is other common functional group such as ketone group, the fungistatic effect is poor.

The preferred intermediate compounds represented by the general formula 3 of the present application include:

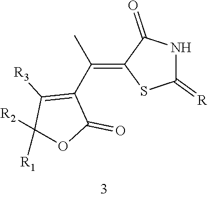

3-1: R₁, R₂ = —(CH₂)₅—, R₃ = CH₃, R = S
3-2: R₁ = R₂ = CH₃, R₃ = CH₃, R = S
3-3: R₁ = R₂ = CH₃, R₃ = Ph, R = NH
3-4: R₁, R₂ = —(CH₂)₅—, R₃ = Ph, R = S
3-5: R₁, R₂ = —(CH₂)₅—, R₃ = 4F—Ph, R = S
3-6: R₁, R₂ = —(CH₂)₅—, R₃ = 4Cl—Ph, R = S
3-7: R₁, R₂ = —(CH₂)₅—, R₃ = 4Br—Ph, R = S
3-8: R₁, R₂ = —(CH₂)₅—, R₃ = 4C₄H₉—Ph, R = S
3-9: R₁, R₂ = —(CH₂)₅—, R₃ = 4OCH₃—Ph, R = S
3-10: R₁, R₂ = —(CH₂)₅—, R₃ = 4SCH₃—Ph, R = S
3-11: R₁, R₂ = —(CH₂)₅—, R₃ = 4SO₂CH₃—Ph, R = S
3-12: R₁, R₂ = —(CH₂)₅—, R₃ = 4CF₃—Ph, R = S
3-13: R₁, R₂ = —(CH₂)₅—, R₃ = 3Br—Ph, R = S
3-14: R₁, R₂ = —(CH₂)₅—, R₃ = 3Cl—Ph, R = S
3-15: R₁, R₂ = —(CH₂)₅—, R₃ = 3F—Ph, R = S
3-16: R₁, R₂ = —(CH₂)₅—, R₃ = 3CF₃—Ph, R = S
3-17: R₁, R₂ = —(CH₂)₅—, R₃ = 3NO₂—Ph, R = S
3-18: R₁, R₂ = —(CH₂)₅—, R₃ = 3OCH₃—Ph, R = S
3-19: R₁, R₂ = —(CH₂)₅—, R₃ = 2Br—Ph, R = S
3-20: R₁, R₂ = —(CH₂)₅—, R₃ = 2Cl—Ph, R = S
3-21: R₁, R₂ = —(CH₂)₅—, R₃ = 2F—Ph, R = S
3-22: R₁, R₂ = —CH₂CH₂CHOCH₃CH₂CH₂—, R₃ = CH₃, R = S
3-23: R₁, R₂ = —CH₂CH₂CHOCH₃CH₂CH₂—, R₃ = Ph, R = S
3-24: R₁, R₂ = —CH₂CH₂CNOCH₃CH₂CH₂—, R₃ = CH₃, R = S
3-25: R₁, R₂ = —CH₂CH₂CNOCH₃CH₂CH₂—, R₃ = Ph, R = S
3-26: R₁, R₂ = —CH₂CH₂CHOHCH₂CH₂—, R₃ = CH₃, R = S
3-27: R₁, R₂ = —CH₂CH₂CHOHCH₂CH₂—, R₃ = CH₃, R = S
3-28: R₁, R₂ = —CH₂CH₂COCH₂CH₂—, R₃ = CH₃, R = NH
3-29: R₁, R₂ = —CH₂CH₂COCH₂CH₂—, R₃ = Ph, R = NH
3-30: R₁, R₂ = —CH₂CH₂CHFCH₂CH₂—, R₃ = CH₃, R = NH
3-31: R₁, R₂ = —CH₂CH₂COCH₂CH₂—, R₃ = Ph, R = NH
3-32: R₁, R₂ = —CH₂CH₂CHClCH₂CH₂—, R₃ = CH₃, R = NH
3-33: R₁, R₂ = —CH₂CH₂CHClCH₂CH₂—, R₃ = CH₃, R = NH
3-34: R₁, R₂ = —CH₂CH₂CHBrCH₂CH₂—, R₃ = CH₃, R = NH
3-35: R₁, R₂ = —CH₂CH₂CHBrCH₂CH₂—, R₃ = Ph, R = NH
3-36: R₁ = R₂ = CH₃, R₃ = CH₃NH, R = NH
3-37: R₁, R₂ = —(CH₂)₅—, R₃ = CH₃NH, R = NH
3-38: R₁ = R₂ = CH₃, R₃ = (CH₃)₂N, R = NH
3-39: R₁, R₂ = —(CH₂)₅—, R₃ = (CH₃)₂N, R = NH
3-40: R₁ = R₂ = CH₃, R₃ = Cl, R = NH
3-41: R₁, R₂ = —(CH₂)₅—, R₃ = Cl, R = NH
3-42: R₁ = R₂ = CH₃, R₃ = F, R = NH
3-43: R₁, R₂ = —(CH₂)₅—, R₃ = F, R = NH
3-44: R₁ = R₂ = CH₃, R₃ = Br, R = NH
3-45: R₁, R₂ = —(CH₂)₅—, R₃ = Br, R = NH
3-46: R₁, R₂ = —(CH₂)₅—, R₃ = C₅H₄N, R = NH
3-47: R₁ = R₂ = CH₃, R₃ = C₅H₄N, R = NH

The present application further provides a preparation method of the aforementioned butenolide compound containing a thiazolidinone structure, wherein the compound represented by the general formula I is synthesized according to the following scheme:

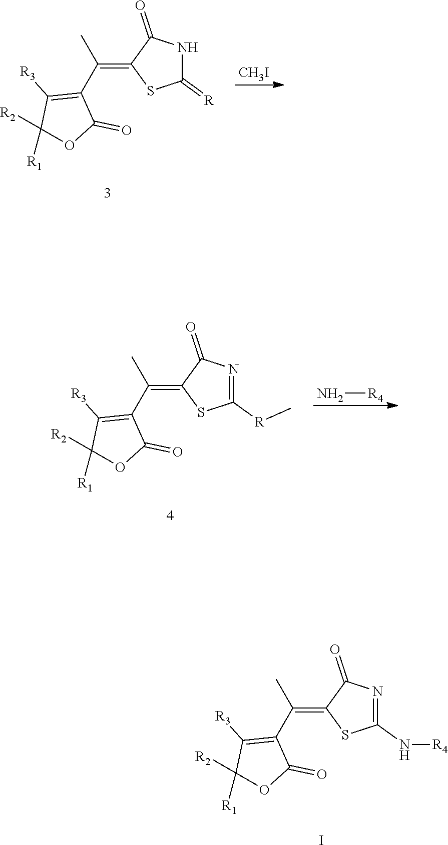

The definitions of R, R₁, R₂, R³, and R₄ in the formula are as described above.

Preferably, the preparation method of the butenolide compounds containing a thiazolidinone structure comprises the following steps:
(1) in a first solvent, subjecting the intermediate compound represented by the general formula 3 to methylation reaction under the action of a base to obtain a compound 4, the base being one or more selected from potassium carbonate, cesium carbonate, sodium hydride, sodium ethoxide, and sodium hydroxide, the first solvent being selected from acetonitrile and/or acetone, and the reaction temperature being raised from room temperature to a reflux temperature; and
(2) in a second solvent, reacting the compound 4 with NH₂—R₄ under reflux in presence of an acid catalyst to obtain the compound represented by the general formula I, the second solvent being selected from toluene and/or xylene, and the acid being one or more selected from oxalic acid, acetic acid, and propionic acid.

Preferably, the base in step (1) is potassium carbonate and/or cesium carbonate; more preferably, the molar ratio of the base to the intermediate compound represented by the general formula 3 is (1.5:1) to (2:1); the molar ratio of iodomethane to the intermediate compound represented by the general formula 3 is (1.2:1) to (1.5:1); the volume molar ratio of the first solvent to the intermediate compound represented by the general formula 3 is (190-210) mL: 20 mmol.

Preferably, the first solvent in step (1) is acetonitrile.

Preferably, the methylation reaction temperature in step (1) is the reflux temperature.

Preferably, the acid in step (2) is oxalic acid and/or acetic acid: more preferably, the molar ratio of the acid to the compound 4 is (1.2:1) to (1.5:1).

Preferably, the second solvent in step (2) is toluene.

Preferably, the reflux temperature in step (2) is 110° C. to 120° C.

The present application also provides a preparation method of the intermediate of butenolide compounds containing a thiazolidinone structure, and the synthesis scheme is as follows:

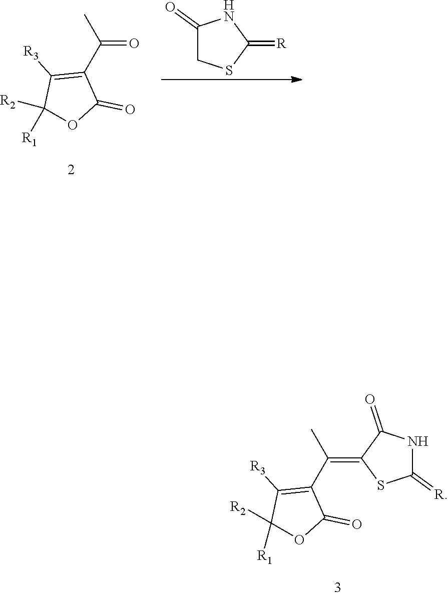

The definitions of R, R₁, R₂, and R₃ in the formula are as described above.

Preferably, the compound 2 is prepared from a compound 1, and the synthesis scheme of the intermediates of butenolide compounds containing a thiazolidinone structure is as follows:

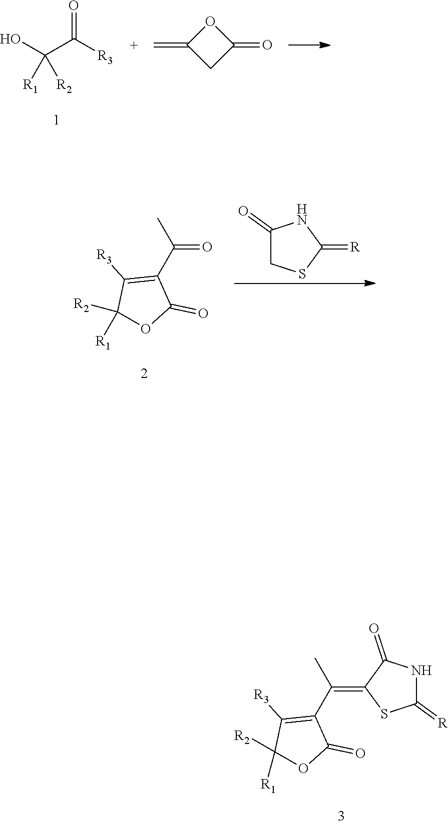

Preferably, the preparation method of the intermediates of the butenolide compounds containing a thiazolidinone structure comprises:
1) compound 1 with diketene in the presence of a catalyst to obtain 3-acetylbutenolide compound 2, the catalyst being selected from triethylamine and/or diisopropylethylamine; and
2) in a third solvent, reacting the compound 2 with

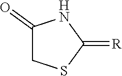

under the action of an organic base under reflux to obtain the intermediate compound represented by the general formula 3, wherein, R is one selected from NH and S: the organic base is one or more selected from ethanolamine, piperidine, pyridine, sodium acetate, and ammonium acetate, and the third solvent is selected from toluene and/or xylene.

Preferably, the catalyst mentioned in step 1) is triethylamine.

Preferably, the organic base mentioned in step 2) is ethanolamine and/or ammonium acetate: more preferably, the molar ratio of the organic base to compound 2 is (3:1) to (5:1).

Preferably, the third solvent in step 2) is toluene: more preferably, the volume molar ratio of toluene to compound 2 is (190-210) mL: 20 mmol.

Preferably, the reflux temperature in step 2) is 110° C. to 120° C.

The present application also provides use of the butenolide compound containing a thiazolidinone structure and the intermediate of the butenolide compound containing a thiazolidinone structure in the preparation of a fungicide.

Preferably, the dosage form of the fungicide is at least one of emulsifiable concentrate, wettable powder, suspension concentrate, dustable powder, soluble powder, aqueous solution, water dispersible granule, smoke generator, granule, and seed dressing agent.

The examples of diseases mentioned below are only used to illustrate the present application, but do not limit the present application in any way.

The compounds represented by the general formula I and the intermediate compounds represented by the general formula 3 can be used to prevent and control the following diseases: oomycetes diseases, such as downy mildew (cucumber downy mildew; rape downy mildew; soy bean downy mildew; sugar beet downy mildew; sugarcane downy mildew; tobacco downy mildew; pea downy mildew; luffa downy mildew; wax gourd downy mildew; muskmelon downy mildew; cabbage downy mildew; spinach downy mildew; radish downy mildew; grape downy mildew, onion downy mildew), white rust (rape white rust, cabbage white rust), damping-off (rape damping-off, tobacco damping-off, tomato damping-off, pepper damping-off, eggplant damping-off, cucumber damping-off, cotton seedling damping-off), pythium rot (pepper pythium rot, luffa pythium rot, wax gourd pythium rot), phytophthora disease (fava bean phytophthora disease, cucumber phytophthora disease, pumpkin phytophthora disease, wax gourd phytophthora disease, watermelon phytophthora disease, muskmelon phytophthora disease, pepper phytophthora disease. Chinese chive phytophthora disease, garlic phytophthora disease, cotton phytophthora disease), and late blight (potato late blight, tomato late blight): diseases caused by Deuteromycetes, such as fusarium wilt (sweet potato fusarium wilt, cotton fusarium wilt, sesame fusarium wilt, castor fusarium wilt, tomato fusarium wilt, bean fusarium wilt, cucumber fusarium wilt, luffa fusarium wilt, pumpkin fusarium wilt, wax gourd fusarium wilt, watermelon fusarium wilt, muskmelon fusarium wilt, pepper fusarium wilt, fava bean fusarium wilt, rape fusarium wilt, soybean fusarium wilt), root rot disease (pepper root rot disease, eggplant root rot disease, bean root rot disease, cucumber root rot disease, balsam pear root rot disease, cotton black root rot disease, fava bean root rot disease), wilt disease (cotton rhizoctoniosis, sesame rhizoctonia rot, pepper rhizoctonia rot, cucumber rhizoctonia rot, cabbage rhizoctonia rot), anthracnose (sorghum anthracnose, cotton anthracnose, kenaf anthracnose, jute anthracnose, flax anthracnose, tobacco anthracnose, mulberry anthracnose, pepper anthracnose, eggplant anthracnose, fava bean anthracnose, cucumber anthracnose, balsam pear anthracnose, zucchini anthracnose, wax gourd anthracnose, watermelon anthracnose, muskmelon anthracnose, lychee anthracnose), verticillium wilt (cotton verticillium wilt, sunflower verticillium wilt, tomato verticillium wilt, pepper verticillium wilt, eggplant verticillium wilt), scab disease (zucchini scab, wax gourd scab, muskmelon scab), gray mold (cotton boll gray mold, kenaf gray mold, tomato gray mold, pepper gray mold, bean gray mold, celery gray mold, spinach gray mold, kiwifruit gray mold), brown spot (cotton brown spot, jute brown spot, beet brown spot, peanut brown spot, pepper brown spot, wax gourd brown spot, soybean brown spot, sunflower brown spot, pea brown spot, fava bean brown spot), black spot (flax false black spot, rape black spot, sesame black spot, sunflower black spot, castor black spot, tomato black spot, pepper black spot, eggplant black spot, bean black spot, cucumber black spot, celery black spot, carrot black spot, apple black spot, peanut black spot), spot blight (tomato spot blight, pepper spot blight, celery spot blight), early blight (tomato early blight, pepper early blight, eggplant early blight, potato early blight, celery early blight), ring rot (Botryosphaeria dothidea) (soy bean ring rot, sesame ring rot, bean ring rot), leaf blight (sesame leaf blight, sunflower leaf blight, watermelon leaf blight, muskmelon leaf blight), stem rot (tomato stem rot, bean stem rot), and other diseases (wheat scab, rice bakanae, northern corn leaf spot, kenaf stem lodging, rice blast disease, chestnut black sheath disease, sugarcane eye spot disease, cotton boll aspergillosis, peanut crown rot disease, soybean stem blight disease, soybean black spot disease, muskmelon Alternaria leaf blight, peanut web blotch, tea red leaf spot, pepper Phyllosticta blight, wax gourd leaf spot disease, celery black rot, spinach heart rot, kenaf leaf mold, kenaf spot disease, jute stem blight, soy bean purple blotch, sesame leaf spot disease, castor gray leaf spot, tea brown leaf spot disease, eggplant cercospora leaf spot, bean cercospora leaf spot, balsam pear cercospora leaf spot, watermelon spot disease, jute macrophomina blight, sunflower root and stem rot, bean charcoal rot disease, soy bean target spot disease, eggplant corynespora leaf spot, cucumber target spot disease, tomato leaf mold disease, eggplant leaf mold disease, fava bean chocolate spot, and the like): basidiomycete diseases, such as rust disease (wheat stripe rust, wheat stem rust, wheat leaf rust, peanut rust, sunflower rust, sugarcane rust, Chinese chive rust, onion rust, chestnut rust, soybean rust, bean rust), smut (corn head smut, corn smut, sorghum head smut, sorghum loose kernel smut, sorghum covered kernel smut, sorghum columnar smut, foxtail millet kernel smut, sugarcane smut), and other diseases (such as wheat sheath blight, and rice sheath blight): ascomycota diseases, such as powdery mildew (wheat powdery mildew; rape powdery mildew; sesame powdery mildew; sunflower powdery mildew; sugar beet powdery mildew, eggplant powdery mildew; pea powdery mildew, luffa powdery mildew; pumpkin powdery mildew, zucchini powdery mildew; wax gourd powdery mildew; muskmelon powdery mildew; grape powdery mildew; fava bean powdery mildew), sclerotinia disease (flax sclerotinia disease, rape sclerotinia disease, soybean sclerotinia disease, peanut sclerotinia disease, tobacco sclerotinia disease, pepper sclerotinia disease, eggplant sclerotinia disease, bean sclerotinia disease, pea sclerotinia disease, cucumber sclerotinia disease, balsam pear sclerotinia disease, wax gourd sclerotinia disease, watermelon sclerotinia disease, celery sclerotinia disease), scab disease (apple scab, pear scab), and the like.

Based on the above technical solutions, the beneficial effects of the present application are as follows:

The present application significantly improves the fungicidal activity and effect of compounds by introducing a thiazolidinone structure into the butenolide skeleton structure, which can effectively protect important crops and livestock in agriculture and horticulture, as well as the environment that humans rely on for survival from pathogen invasion.

### BRIEF DESCRIPTION OF THE DRAWINGS

In order to more clearly illustrate the technical solutions of the present application or the prior art, a brief introduction will be given to the accompanying drawings required for the description of examples or the prior art. It is obvious that the accompanying drawings described below are some examples of the present application. For a person skilled in the art, other drawings can be obtained based on these drawings without creative labor.

FIG. 1 shows the EC₅₀ determination plate photos of compounds 3-1 (Graph A) and 3-14 (Graph B) provided by the present application against wheat scab pathogen fungi: wherein, in Graph A, from left to right in the first row; the concentrations of compound 3-1 are 1.0 mg/L, 0.25 mg/L, and 0.063 mg/L, respectively: the concentrations of compound 3-1 in the second row from left to right are 0.016 mg/L and 0.0040 mg/L, respectively, and the last plate is the CK control group. In Graph B, from left to right in the first row; the first plate is the CK control group, the concentrations of compound 3-14 in the remaining plates are 50 mg/L and 12.5 mg/L, respectively; and from left to right in the second row; the concentrations of compound 3-14 are 3.13 mg/L, 0.78 mg/L, and 0.20 mg/L, respectively.

FIG. 2 shows the EC₅₀ determination plate photos of phenamacril (Graph A) and compound 3-5 (Graph B) provided by the present application against wheat scab pathogen fungi: wherein, in Graph A, from left to right in the first row; the first plate is the CK control group, and the concentrations of phenamacril in the remaining plates are 10 mg/L and 2.5 mg/L, respectively: from left to right in the second row; the concentrations of phenamacril are 0.63 mg/L, 0.16 mg/L, and 0.039 mg/L, respectively: in Graph B, from left to right in the first row; the first plate is the CK control group, the concentrations of compound 3-5 in the remaining plates are 5.0 mg/L and 1.3 mg/L, respectively; and from left to right in the second row; the concentrations of compound 3-5 are 0.31 mg/L, 0.075 mg/L, and 0.019 mg/L, respectively.

FIG. 3 shows the EC₅₀ determination plate photos of phenamacril (Graph A) and compound 3-1 (Graph B) provided by the present application against rice bakanae pathogen fungi: wherein, in Graph A, from left to right in the first row; the concentrations of phenamacril are 5.0 mg/L, 0.31 mg/L, and 0.075 mg/L, respectively: from left to right in the second row; the first plate is the CK control group, and the concentrations of phenamacril in the remaining plates are 1.3 mg/L and 0.019 mg/L, respectively: in Graph B, from left to right in the first row; the concentrations of compound 3-1 are 1.0 mg/L, 0.25 mg/L, and 0.063 mg/L, respectively; and from left to right in the second row; the concentrations of compound 3-1 are 0.016 mg/L and 0.0040 mg/L, respectively, and the last plate is the CK control group.

FIG. 4 shows the EC₅₀ determination plate photos of compounds 3-5 (Graph A) and 3-22 (Graph B) against rice bakanae pathogen fungi: wherein, in Graph A, from left to right in the first row; the concentrations of compound 3-5 are 4.0 mg/L, 1.0 mg/L, and 0.25 mg/L, respectively: from left to right in the second row; the first plate is the CK control group, and the concentrations of compound 3-5 in the remaining plates are 2.0 mg/L and 0.5 mg/L, respectively: in Graph B, from left to right in the first row; the concentrations of compound 3-22 are 4.0 mg/L, 1.0 mg/L, and 0.25 mg/L, respectively; and from left to right in the second row; the concentrations of compound 3-22 are 0.063 mg/L and 0.016 mg/L, respectively, and the last plate is the CK control group.

FIG. 5 shows the determination plate photos of 50 mg/L phenamacril and compound 3-1 against resistant strains T2-5, T10, and T32.

FIG. 6 shows the determination plate photos of 50 mg/L phenamacril and compound 3-1 against resistant strains T18, T48, and T79.

FIG. 7 shows the determination plate photos of compound 3-1 at low concentrations (0.5 mg/L and 1 mg/L) against standard strain 2021, resistant strain T10, and resistant strain T2-5.

FIG. 8 shows the determination plate photos of compound 3-1 at low concentrations (0.5 mg/L and 1 mg/L) against resistant strains T32, T13, and T12.

### SPECIFIC MODES FOR CARRYING OUT THE EMBODIMENTS

In order to clarify the purpose, technical solution, and advantages of the present application, the technical solution of the present application will be described clearly and completely in conjunction with the accompanying drawings. Obviously, the described examples are a part of the examples of the present application, not all of them. Based on the examples of the present application, all other examples obtained by ordinary skilled persons in the art without creative labor are within the scope of protection of the present application.

The following Examples are used to illustrate the present application, but not to limit the scope of the present application.

Unless otherwise specified, all raw materials used below are commercially available.

### Example 1: Preparation of Intermediate Compounds Represented by General Formula 3

The present Example provides an intermediate: 5-(4-methyl-2-oxo-1-oxaspiro[4,5]-dec-3-en-3-ethylidene)-2-thio-4-thiazolidinone, the preparation method thereof comprises:

Raw materials 2-thio-4-thiazolidinone (1.276 g, 11 mmol) and ammonium acetate (2.310 g, 30 mmol) were dissolved in 100 mL of toluene, and then 4-methyl-3-acetyl-2-oxo-1-oxaspiro[4,5]-dec-3-ene (compound 2) (2.080 g, 10 mmol) was added thereto, to react at 110° C. for 15 h under refluxing and stirring. After the reaction was completed (monitored by TLC), the solvent was removed by vacuum rotary evaporation, and then ethyl acetate and water were added for extraction. The organic phase was washed with saturated sodium chloride solution, dried with anhydrous sodium sulfate, filtered and vacuum desolventized, and the residue was separated by silica gel column chromatography (eluent is ethyl acetate and petroleum ether (boiling range 60° C. to 90° C.) at a volume ratio of 1:6) to obtain 1.807 g of a yellow solid with a melting point of 118° C. to 119° C. and a yield of 56%.

¹H NMR (300 MHz, CDCl₃) δ: 10.31 (brs, 1H), 2.53, 2.13 (s, 3H), 1.96, 1.91 (s, 3H), 1.88-1.46 (m, 10H).

In the present application, other intermediate compounds represented by general formula 3 can also be obtained by performing corresponding replacement with different raw materials, which are not listed one by one here.

### Example 2: Preparation of a Compound Represented by General Formula I

The present Example provides a compound: (E)-2-(3′,4′-dichloro-5-fluoro-[1,1′-biphenyl]-2-amino)-5-(4-phenyl-2-oxo-1-oxaspiro[4,5]-dec-3-en-3-ethylidene)-thiazol-4-one, the preparation method thereof comprises:

(1) 5-(4-phenyl-2-oxo-1-oxaspiro[4,5]-dec-3-en-3-ethylidene)-2-thio-4-thiazolidinone (3.850 g, 10 mmol) was dissolved in 100 mL of acetonitrile, potassium carbonate (2.070 g, 15 mmol) and iodomethane (1.704 g, 12 mmol) were added to react at room temperature for 8 h under stirring. After the reaction was completed (monitored by TLC), the potassium carbonate was removed by vacuum filtration, the solvent was removed by vacuum rotary evaporation, and the residue was subjected to recrystallization with ethyl acetate to obtain 3.352 g of (E)-2-methylthio-5-(4-phenyl-2-oxo-1-oxaspiro[4,5]-dec-3-en-3-ethylidene)-thiazol-4-one (compound 4) as a yellow solid with a melting point 141° C. to 143° C. and a yield of 84%. 1H NMR (300 MHz, CDCl₃) δ: 7.41-7.33 (m, 3H), 7.18-7.11 (m, 2H), 2.72 (s, 3H), 2.11-2.00 (m, 1H), 1.91 (s, 3H), 1.90-1.64 (m, 8H), 1.54-1.44 (m, 1H). ¹³C NMR (75 MHz, CDCl₃) δ: 190.52, 176.08, 169.04, 166.82, 137.92, 132.05, 131.89, 129.40, 129.11, 128.81, 127.34, 88.96, 33.41, 33.13, 26.14, 24.64, 22.22, 15.83.

(2) (E)-2-methylthio-5-(4-phenyl-2-oxo-1-oxaspiro[4,5]-dec-3-en-3-ethylidene)-thiazol-4-one (0.399 g, 1 mmol) was dissolved in a mixed solution of 1.5 mL of glacial acetic acid and 13.5 mL of toluene, 3′,4′-dichloro-5-fluoro-[1,1′-biphenyl]-2-amine (0.383 g, 1.5 mmol) was added to react at 110° C. for 12 h under refluxing and stirring. After the reaction was completed (monitored by TLC), the solvent was removed by vacuum rotary evaporation, and the residue was separated by silica gel column chromatography (eluent is ethyl acetate and petroleum ether (boiling range 60° C. to 90° C.) at a volume ratio of 1:6) to obtain 0.551 g of a red solid with a melting point of 161° C. to 163° C. and a yield of 91%.

¹H NMR (300 MHz, CDCl₃) δ: 10.16 (brs, 1H), 7.49-7.29 (m, 5H), 7.20-6.88 (m, 6H), 2.00-0.96 (m, 10H), 1.72 (s, 3H). ¹³C NMR (75 MHz, CDCl₃) δ: 169.54, 166.52, 165.17, 160.40 (d, ¹J_{CF}=243.9 Hz), 151.35, 140.86 (d, ⁴J_{CF}=2.7 Hz), 138.01, 134.40, 134.29, 132.29, 131.93, 131.77, 130.92, 130.25, 129.46, 129.09, 128.90, 128.63, 127.35, 125.97, 123.22 (d, ³J_{CF}=8.3 Hz), 117.13 (d, ²J_{CF}=22.9 Hz), 115.78 (d, ²J_{CF}=22.1 Hz), 89.01, 33.39, 33.00, 24.63, 22.83, 22.20. HRMS m/z: C₃₂H₂₅Cl₂FN₂O₃S 607.1022 [M+H]⁺ (calcd [M+H]⁺607.1020).

### Example 3: Preparation of a Compound Represented by General Formula I

The present Example provides a compound: (E)-2-(2′,3′,4′-trifluoro-[1,1′-biphenyl]-2-amino)-5-(4-methyl-2-oxo-1-oxaspiro[4,5]-dec-3-en-3-ethylidene)-thiazol-4-one, the preparation method thereof comprises:

(1) the intermediate 5-(4-methyl-2-oxo-1-oxaspiro[4,5]-dec-3-en-3-ethylidene)-2-thio-4-thiazolidinone (3.230 g, 10 mmol) was dissolved in 100 mL of acetonitrile, potassium carbonate (2.070 g, 15 mmol) and iodomethane (1.704 g, 12 mmol) were added to react at room temperature for 8 h under stirring. After the reaction was completed (monitored by TLC), the potassium carbonate was removed by vacuum filtration, the solvent was removed by vacuum rotary evaporation, and the residue was subjected to recrystallization with ethyl acetate to obtain 2.966 g of (E)-2-methylthio-5-(4-methyl-2-oxo-1-oxaspiro[4,5]-dec-3-en-3-ethylidene)-thiazol-4-one as a yellow solid with a melting point of 142° C. to 144° C. and a yield of 88%. ¹H NMR (300 MHz, CDCl₃) δ: 2.72 (s, 3H), 2.56, 2.22 (s, 3H), 1.93, 1.86 (s, 3H), 1.88-1.67 (m, 9H), 1.30-1.19 (m, 1H).

(2) (E)-2-methylthio-5-(4-methyl-2-oxo-1-oxaspiro[4,5]-dec-3-en-3-ethylidene)-thiazol-4-one (0.337 g, 1 mmol) was dissolved in a mixed solution of 1.5 mL of glacial acetic acid and 13.5 mL of toluene, 2′,3′,4′-trifluoro-[1,1′-biphenyl]-2-amine (0.335 g, 1.5 mmol) was added to react at 110° C. for 12 h under refluxing and stirring. After the reaction was completed (monitored by TLC), the solvent was removed by vacuum rotary evaporation, and the residue was separated by silica gel column chromatography (eluent being ethyl acetate and petroleum ether (boiling range 60° C. to 90° C.) at a volume ratio of 1:6) to obtain 0.440 g of an orange solid with a melting point of 138° C. to 139° C. and a yield of 86%. ¹H NMR (300 MHz, CDCl₃) δ: 9.86 (brs, 1H), 7.43-7.17 (m, 3H), 7.05-6.80 (m, 3H), 2.48, 2.02 (s, 3H), 1.87, 1.86 (s, 3H), 1.85-1.41 (m, 10H).

### Example 4: Preparation of a Compound Represented by General Formula I

The present Example provides a compound: (E)-2-(4′-trifluoromethoxy-[1,1′-biphenyl]-2-amino)-5-(4-methyl-2-oxo-1-oxaspiro[4,5]-dec-3-en-3-ethylidene)-thiazol-4-one, the preparation method thereof comprises:
(1) (E)-2-methylthio-5-(4-methyl-2-oxo-1-oxaspiro[4,5]-dec-3-en-3-ethylidene)-thiazol-4-one was prepared by using step (1) of Example 3:
(2) (E)-2-methylthio-5-(4-methyl-2-oxo-1-oxaspiro[4,5]-dec-3-en-3-ethylidene)-thiazol-4-one (0.337 g, 1 mmol) was dissolved in a mixed solution of 1.5 mL of glacial acetic acid and 13.5 mL of toluene, 4′-trifluoromethoxy-[1,1′-biphenyl]-2-amine (0.380 g, 1.5 mmol) was added to react at 110° C. for 14 h under refluxing and stirring. After the reaction was completed (monitored by TLC), the solvent was removed by vacuum rotary evaporation, and the residue was separated by silica gel column chromatography (eluent being ethyl acetate and petroleum ether (boiling range 60° C. to 90° C.) at a volume ratio of 1:6) to obtain 0.493 g of a yellow solid with a melting point of 146° C. to 148° C. and a yield of 91%. ¹H NMR (300 MHz, CDCl₃) δ: 9.81 (brs, 1H), 7.41-7.08 (m, 7H), 7.02-6.87 (m, 3=1H), 2.48, 2.02 (s, 3H), 1.85 (s, 3H), 1.84-1.41 (m, 10H). HRMS m/z: C₂₈H₂₅F₃N₂O₄S 543.1562 [M+H]⁺ (calcd [M+H]⁺ 543.1560).

In the present application, other compounds represented by general formula I can also be obtained by performing corresponding replacement with different raw materials, which are not listed one by one here.

### Experimental Example

### 1. Fungicidal Activity Determination

In vitro fungicidal activity or in vivo protective effect tests were conducted on various fungal diseases of plants using the compound samples of the present application. The results of fungicidal activity determination are shown in the following examples.

### (1) In Vitro Fungicidal Activity Determination

The determination method is as follows: This experiment was conducted according to the agricultural industry standard of the People's Republic of China (NY/T1156.2-2006), using the mycelial growth rate method for determination.

A certain amount of the raw agent was weighed with an analytical balance, DMSO as the dissolution solvent was added, the test compound was prepared into a solution with a mass concentration of 5000 mg/L by dilution to a constant volume, then the solution was diluted with a potato dextrose agar (PDA) liquid medium to prepare a 50 mg/L toxic PDA plate for testing. Various cultivated pathogenic bacteria were inoculated into the center of an agent-containing PDA medium plate under sterile operating conditions, with the mycelial side facing down, the plate was covered with a lid and placed in an incubator to incubate in the dark at 24° C. for 2-3 days. The pathogen mycelium growth situation was investigated based on the growth situation of colonies in the blank control culture dish. After the colonies in the blank control had fully grown, the diameter of each treated colony was measured using the cross-validation method. Each sample was measured in parallel for three times, and the average value was taken. Carbendazim, thifluzamide, and phenamacril were used as control agents. The mycelial growth inhibition rate of each agent treatment against various pathogens was calculated based on the ratio of the difference between the colony growth diameter of blank control and the agent-treated colony growth diameter to the colony growth diameter of blank control, as shown in Table 2.

TABLE 2
In vitro fungicidal activity of some intermediates and compounds (inhibition rate/%)

| | Pathogenic | Pathogenic | | Pathogenic | Pathogenic | Pathogenic |
|---|---|---|---|---|---|---|
| | fungi of | fungi of pepper | Pathogenic | fungi of | fungi of | fungi of |
| | rice sheath | phytophthora | fungi of | rice bakanae | rape | tomato |
| Compounds | blight | disease | wheat scab | disease | sclerotinia | gray mold |
| 3-1 | 45.2 ± 2.1 | 59.7 ± 0.4 | 100.0 ± 0.0 | 100.0 ± 0.0 | 82.4 ± 1.7 | 62.4 ± 2.5 |
| 3-2 | 42.4 ± 5.0 | 76.7 ± 2.4 | 51.7 ± 4.1 | 31.7 ± 0.9 | 39.6 ± 3.1 | 29.5 ± 4.5 |
| 3-4 | 31.9 ± 5.6 | 44.8 ± 0.3 | 27.7 ± 2.9 | 15.8 ± 2.4 | 68.3 ± 4.2 | 33.6 ± 1.7 |
| 3-5 | 56.0 ± 3.3 | 42.5 ± 3.1 | 100.0 ± 0.0 | 86.9 ± 4.4 | 88.8 ± 1.2 | 65.0 ± 5.1 |
| 3-14 | 56.4 ± 0.5 | 37.5 ± 2.1 | 87.0 ± 3.0 | 79.7 ± 5.0 | 86.9 ± 3.4 | 63.6 ± 0.2 |
| 3-15 | 47.6 ± 2.1 | 35.0 ± 2.1 | 100.0 ± 0.0 | 83.3 ± 0.4 | 86.0 ± 2.1 | 64.0 ± 2.3 |
| 3-17 | 44.8 ± 2.7 | 43.3 ± 1.3 | 100.0 ± 0.0 | 88.5 ± 1.7 | 84.8 ± 0.7 | 54.3 ± 1.8 |
| 3-22 | 32.4 ± 0.8 | 45.8 ± 1.6 | 100.0 ± 0.0 | 100.0 ± 0.0 | 61.2 ± 4.8 | 48.2 ± 1.2 |
| 3-24 | 51.5 ± 3.4 | 28.1 ± 2.6 | 100.0 ± 0.0 | 100.0 ± 0.0 | 63.0 ± 3.3 | 63.1 ± 5.3 |
| 3-26 | 11.8 ± 5.5 | 54.2 ± 2.5 | 46.7 ± 5.0 | 25.3 ± 2.0 | 9.0 ± 1.7 | 3.9 ± 1.4 |
| 3-46 | 50.3 ± 1.4 | 66.8 ± 2.7 | 70.2 ± 1.3 | 68.7 ± 3.5 | 89.6 ± 0.5 | 51.7 ± 3.4 |
| I-1-10 | 55.4 ± 1.7 | 25.4 ± 3.6 | 5.7 ± 1.2 | 60.7 ± 1.6 | 55.7 ± 3.1 | 35.4 ± 1.7 |
| I-1-11 | 20.1 ± 1.8 | 34.9 ± 0.9 | 49.3 ± 2.0 | 35.7 ± 0.9 | 45.2 ± 3.4 | 65.3 ± 5.9 |
| I-1-12 | 36.9 ± 1.5 | 15.7 ± 1.8 | 22.8 ± 2.6 | 15.2 ± 4.1 | 36.5 ± 3.0 | 54.2 ± 0.2 |
| I-1-13 | 27.8 ± 2.4 | 33.6 ± 2.9 | 8.7 ± 3.1 | 33.6 ± 0.7 | 55.7 ± 1.6 | 15.4 ± 2.4 |
| I-1-14 | 55.4 ± 2.8 | 26.4 ± 1.8 | 14.5 ± 4.2 | 25.8 ± 1.2 | 36.9 ± 2.4 | 11.2 ± 1.2 |
| I-1-15 | 15.2 ± 4.1 | 8.8 ± 1.5 | 27.3 ± 0.5 | 23.5 ± 1.4 | 44.5 ± 1.2 | 3.7 ± 5.2 |
| I-1-16 | 18.7 ± 2.4 | 2.5 ± 1.8 | 28.7 ± 1.5 | 20.5 ± 2.4 | 12.5 ± 3.1 | 13.2 ± 2.6 |
| I-1-17 | 12.4 ± 1.6 | 13.0 ± 2.7 | 29.2 ± 1.8 | 22.8 ± 2.6 | 11.4 ± 2.1 | 11.3 ± 2.8 |
| I-1-18 | 15.6 ± 0.5 | 0.4 ± 1.8 | 18.2 ± 4.4 | 12.5 ± 2.4 | 5.7 ± 0.7 | 9.1 ± 3.5 |
| I-5-11 | 44.5 ± 1.5 | 11.8 ± 2.1 | 100.0 ± 0.0 | 90.2 ± 1.4 | 67.5 ± 0.6 | 15.7 ± 1.1 |
| I-9-5 | 40.1 ± 1.1 | 15.9 ± 0.4 | 100.0 ± 0.0 | 88.7 ± 3.1 | 89.1 ± 1.5 | 25.9 ± 2.1 |
| I-15-13 | 22.5 ± 1.4 | 22.1 ± 1.6 | 84.5 ± 2.0 | 100.0 ± 0.0 | 55.4 ± 1.9 | 19.5 ± 1.4 |
| I-20-3 | 32.4 ± 0.9 | 18.4 ± 2.0 | 100.0 ± 0.0 | 89.0 ± 3.0 | 90.1 ± 3.5 | 26.7 ± 2.3 |
| Carbendazim | 100 ± 0 | 4.6 ± 1.2 | 100 ± 0 | 87.5 ± 3.1 | 72.5 ± 2.1 | 5.4 ± 2.4 |
| Thifluzamide | 90.1 ± 4.5 | 15.1 ± 2.8 | 13.4 ± 6.3 | 5.8 ± 1.4 | 83.0 ± 1.2 | 28.2 ± 1.2 |
| Phenamacril | 11.5 ± 1.4 | 8.1 ± 2.1 | 100.0 ± 0.0 | 100.0 ± 0.0 | 23.0 ± 1.3 | 13.1 ± 0.3 |

As shown in the table above, at a concentration of 50 mg/L, the compounds and intermediates exhibit high inhibition rates against pathogenic fungi of wheat scab, pathogenic fungi of rice sheath blight, pathogenic fungi of rape sclerotinia and the like, and some compounds can even completely inhibit the growth of pathogenic fungi, have excellent fungi inhibiting activity.

### 2. Toxicity Testing

Precise toxicity testing was performed on compounds with initial screening inhibition rates greater than 70%. PDA medium was used to dilute the compounds to be tested into five concentration gradients of toxic plates and inoculate with corresponding pathogenic fungi, respectively. Each concentration gradient was repeated for three times, and the EC₅₀ and EC₉₀) (mg/L) of the compounds were obtained using SPSS software, as shown in Table 3.

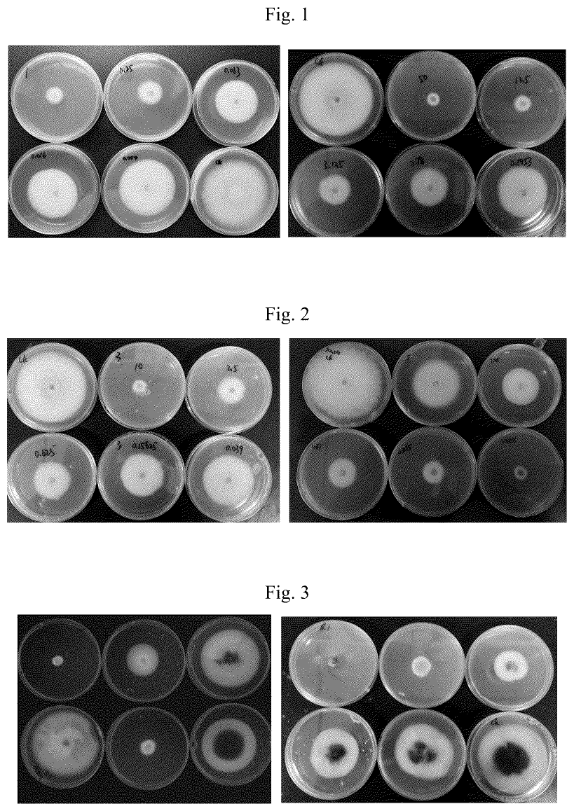

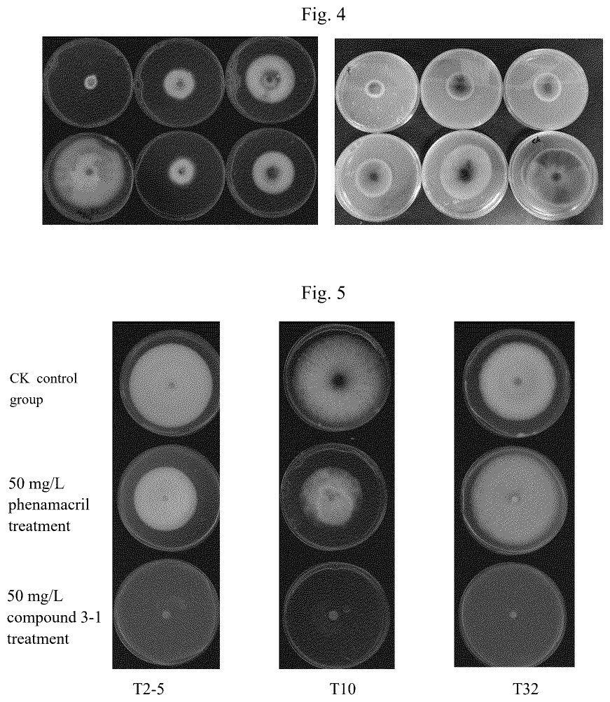

TABLE 3
In vitro fungi inhibiting activity EC₅₀ and EC₉₀ (mg/L) of some
intermediates and compounds against plant pathogenic fungi

| | | | | | | Toxicity | |
|---|---|---|---|---|---|---|---|
| Compounds | Pathogenic fungi | EC₅₀ (mg/L) | | EC₉₀ (mg/L) | | regression equation | R² |
| 3-1 | Wheat scab | 0.079 | (0.06-0.10) | 4.97 | (3.02-9.21) | y = 0.709x + 5.7878 | 0.95 |
| 3-1 | Rice bakanae | 0.72 | (0.60-0.86) | 5.04 | (3.81-7.15) | y = 1.4802x + 5.1963 | 0.96 |
| 3-1 | Rape sclerotinia | 2.02 | (1.45-3.12) | 9.81 | (5.65-23.01) | y = 1.7761x + 4.5188 | 0.91 |
| 3-2 | Pepper phytophthora | 16.64 | (9.81-35.42) | 168.41 | (98.74-456.78) | y = 1.2919x + 3.4223 | 0.92 |
| 3-5 | Rice bakanae | 1.26 | (1.13-1.41) | 10.82 | (8.39-14.91) | y = 1.3648x + 4.8639 | 0.97 |
| 3-5 | Wheat scab | 0.42 | (0.33-0.53) | 34.87 | (21.00-65.89) | y = 0.6855x + 5.2695 | 0.95 |
| 3-5 | Rape sclerotinia | 0.27 | (0.20-0.35) | 8.20 | (6.09-11.88) | y = 0.8497x + 5.502 | 0.95 |
| 3-14 | Rice bakanae | 3.27 | (2.81-3.93) | 34.68 | (23.28-58.41) | y = 1.333x + 4.3245 | 0.91 |
| 3-14 | Wheat scab | 1.04 | (0.81-1.30) | 54.96 | (37.28-88.41) | y = 0.7425x + 4.9903 | 0.98 |
| 3-14 | Rape sclerotinia | 5.47 | (3.52-7.43) | 70.71 | (43.23-165.08) | y = 1.1229x + 4.1795 | 0.97 |
| 3-15 | Rice bakanae | 1.51 | (1.17-1.98) | 20.9 | (11.2-58.9) | y = 1.171x + 4.7827 | 0.95 |
| 3-15 | Wheat scab | 1.33 | (1.00-1.80) | 263.84 | (119.64-748.68) | y = 0.5574x + 4.935 | 0.98 |
| 3-15 | Rape sclerotinia | 0.60 | (0.40-0.85) | 10.15 | (6.19-21.00) | y = 1.0278x + 5.210 | 0.91 |
| 3-17 | Rice bakanae | 1.92 | (1.62-2.33) | 15.82 | (10.47-28.01) | y = 1.5607x + 4.5398 | 0.95 |
| 3-17 | Wheat scab | 2.90 | (2.21-3.98) | 27.13 | (13.42-67.02) | y = 0.6524x + 4.6992 | 0.96 |
| 3-17 | Rape sclerotinia | 7.69 | (5.03-10.63) | 44.83 | (28.31-104.60) | y = 1.6593x + 3.5116 | 0.93 |
| 3-22 | Rice bakanae | 0.39 | (0.22-0.61) | 14.52 | (6.51-59.30) | y = 0.9508x + 5.4042 | 0.95 |
| 3-22 | Wheat scab | 1.94 | (1.58-2.41) | 100.42 | (62.20-181.28) | y = 0.7559x + 4.7819 | 0.99 |
| 3-24 | Rice bakanae | 1.53 | (1.10-1.86) | 44.19 | (36.45-116.35) | y = 0.876x + 4.8387 | 0.97 |
| 3-46 | Rape sclerotinia | 0.20 | (0.09-0.51) | 3.07 | (1.25-9.84) | y = 1.0698x + 5.7594 | 0.98 |
| I-5-11 | Wheat scab | 0.40 | (0.36-4.44) | 2.88 | (2.34-3.72) | y = 1.4950x + 5.5870 | 0.98 |
| I-5-11 | Rice bakanae | 1.12 | (1.00-1.25) | 8.52 | (6.80-1.13) | y = 1.4681x + 4.9236 | 0.99 |
| I-9-5 | Wheat scab | 0.46 | (0.42-0.51) | 2.73 | (2.26-3.41) | y = 1.6615x + 5.5537 | 0.99 |
| I-9-5 | Rice bakanae | 1.62 | (1.45-1.82) | 12.41 | (9.62-17.02) | y = 1.4632x + 4.6954 | 0.99 |
| I-9-5 | Rape sclerotinia | 0.34 | (0.30-0.38) | 2.76 | (2.22-3.63) | y = 1.4268x + 5.6694 | 0.99 |
| I-15-13 | Wheat scab | 2.11 | (1.86-2.42) | 19.85 | (14.41-29.85) | y = 1.3555x + 4.5603 | 0.97 |
| I-15-13 | Rice bakanae | 0.15 | (0.14-0.17) | 0.90 | (0.76-1.10) | y = 1.7143x + 6.4061 | 0.98 |
| I-20-3 | Wheat scab | 0.11 | (0.096-0.12) | 0.76 | (0.64-0.94) | y = 1.6296x + 6.5534 | 0.96 |
| I-20-3 | Rice bakanae | 1.79 | (1.60-2.02) | 14.21 | (10.86-19.89) | y = 1.4421x + 4.6346 | 0.99 |
| I-20-3 | Rape sclerotinia | 0.30 | (0.27-0.34) | 1.79 | (1.52-2.20) | y = 1.7100x + 5.8899 | 0.98 |
| Thifluzamide | Rape sclerotinia | 1.53 | (1.20-1.95) | 16.28 | (10.90-27.82) | y = 1.2376x + 4.752 | 0.94 |
| Phenamacril | Wheat scab | 0.078 | (0.01-0.20) | 5.60 | (1.98-45.86) | y = 0.6842x + 5.7625 | 0.97 |
| Phenamacril | Rice bakanae | 0.071 | (0.05-0.08) | 0.54 | (0.38-0.83) | y = 1.5652x + 6.9494 | 0.96 |

Wherein, the EC₅₀ determination plate photos of compounds 3-1 and 3-14 against wheat scab pathogen fungi are shown in FIG. 1: the EC₅₀ determination plate photos of phenamacril and compound 3-5 provided by the present application against wheat scab pathogen fungi are shown in FIG. 2: the EC₅₀ determination plate photos of phenamacril and compound 3-1 provided by the present application against rice bakanae pathogen fungi are shown in FIG. 3; and the EC₅₀ determination plate photos of compounds 3-5 and 3-22 against rice bakanae pathogen fungi are shown in FIG. 4.

In the present invention, other compounds have similar effects as the above-mentioned compounds and are not listed one by one here.

### 3. In Vitro Fungicidal Activity Determination

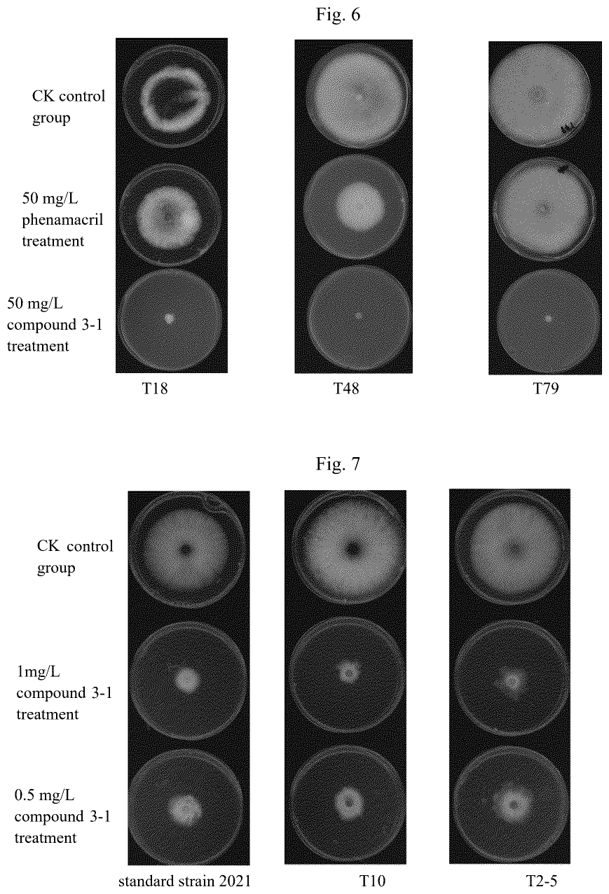

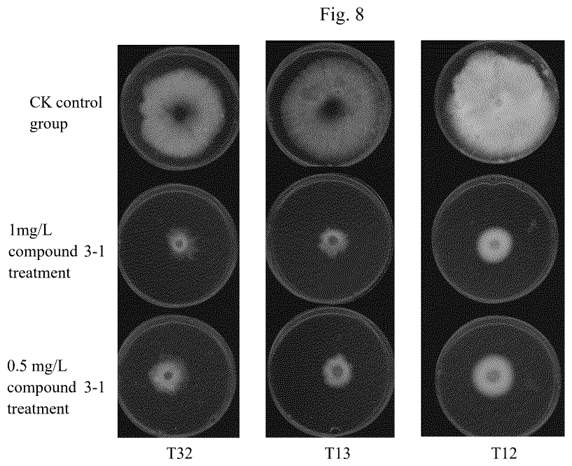

The in vitro fungicidal activity of compound 3-1 was determined using Fusarium graminearum strains resistant to phenamacril, wherein the determination plate photos of high concentration (50 mg/L) of phenamacril and compound 3-1 against resistant strains T2-5, T10, and T32 are shown in FIG. 5, and the determination plate photos of resistant strains T18, T48, and T79 are shown in FIG. 6. Further determination was conducted on standard strain 2021, resistant strain T10, resistant strain T2-5, resistant strain T32, resistant strain T13, and resistant strain T12 with low concentrations (0.5 mg/L and 1 mg/L) of compound 3-1. The plate photos are shown in FIG. 7 and FIG. 8, respectively.

The results show that compound 3-1 can completely inhibit the growth of all resistant strains at a concentration of 50 mg/L. At concentrations of 1 mg/L and 0.5 mg/L, the inhibition rate of this compound on resistant strains was the same as that of the standard wild-type Fusarium graminearum strain, fully demonstrating that this type of compound does not have interactive resistance with phenamacril, which also lays the foundation for subsequent field applications.

The resistant strain numbers are derived from the following literature:
[1] Li, B.; Zheng, Z T.; Liu, X M.; Cai, Y Q.; Mao, X W.; Zhou, M G. Genotypes and Characteristics of Phenamacril-Resistant Mutants in Fusarium asiaticum. Plant Disease, 2016, 100, 1754-1761.

### 4. In Vivo Protective Activity Determination

The determination method was as follows: the live pot cultivation determination method was used, that is, the compound sample to be tested was dissolved in a small amount of solvent (the type of solvent was such as acetone, methanol, and DMF, and was selected according to its dissolving ability for the sample, with a volume ratio of solvent amount to spray amount equal to or less than 0.05), diluted with water containing 0.1% Tween 80, and prepared into a test solution with a required concentration. With a crop sprayer, the test solution was sprayed on a pathogen host plant (the host plant was a standard potted seedling cultivated in a greenhouse), and inoculation with the pathogen was performed after 24 hours. Based on the characteristics of the pathogen, plants that require temperature control and moisturizing cultivation were inoculated and placed in an artificial climate chamber for cultivation, and transferred to a greenhouse for cultivation after the pathogen infection was completed. Plants that do not require moisturizing cultivation were directly placed in a greenhouse, inoculated and cultivated. The disease prevention efficacy of the compound was evaluated after the disease had fully developed (usually one week).

At a concentration of 400 mg/L, the in vivo protective activity determination results of some compounds are as follows:

Compound I-7-5 has an efficacy of 95% against cucumber downy mildew; and compound I-12-15 has an efficacy of 93% against cucumber downy mildew:

Compound I-6-9 has an efficacy of 88% against wheat powdery mildew; compound I-14-6 has an efficacy of 85% against wheat powdery mildew; and compound I-16-7 has an efficacy of 81% against wheat powdery mildew:

Compound I-3-5 has an efficacy of 86% against corn rust disease, and compound I-10-17 has an efficacy of 89% against corn rust disease.

According to the above biological activity determination method, corresponding tests were conducted on other compounds represented by general formula I and key intermediates—other intermediate compounds represented by general formula 3 of the present application, which also had activity equivalent to the listed compounds, and were not listed one by one here.

Finally, it should be noted that the above Examples are only used to illustrate the technical solution of the present application, and not to limit the present application. Although the present application has been described in detail with reference to the aforementioned Examples, a person skilled in the art should understand that they can still modify the technical solutions described in the aforementioned examples, or equivalently replace some of the technical features therein, and these modifications or substitutions do not cause the nature of the corresponding technical solutions to deviate from the spirit and scope of the technical solution of each example of the present application.

### INDUSTRIAL APPLICABILITY

The present application provides butenolide compounds containing a thiazolidinone structure as well as a preparation method therefor and the use thereof. Butenolide compounds containing a thiazolidinone structure, characterized by having the following general formula:

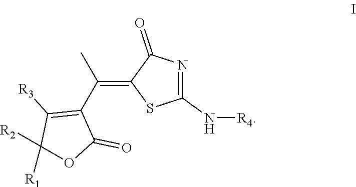

The compounds described in the present application exhibit excellent fungicidal activity against various pathogens in agriculture or other fields, which can effectively protect important crops and livestock in agriculture and horticulture, as well as the environment that human beings rely on for survival from invasion of pathogens, with good economic value and application prospects.

1. A butenolide compound containing a thiazolidinone structure, represented by the following general formula: 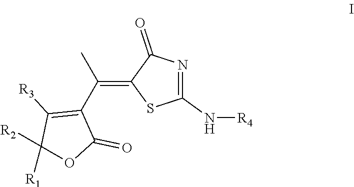
in the formula:
R₁ and R₂ are each independently selected from hydrogen and C₁-C₁₂ alkyl; or, R₁ and R₂, together with the carbon atom bonded therewith, form C₃-C₁₂ cycloalkyl or C₃-C₁₂ cyclic heteroalkyl, heteroatom in the heteroalkyl is N, O, or S, wherein the hydrogen in the C₃-C₁₂ cycloalkyl or the C₃-C₁₂ cyclic heteroalkyl may be monosubstituted or polysubstituted by R₅;
R₃ is selected from hydrogen, hydroxyl, C₁-C₁₂ alkyl, C₅-C₇ cycloalkyl, unsubstituted aryl or heteroaryl, or aryl or heteroaryl containing one to three R₆ substituents;
R₄ is selected from C₁-C₁₂ alkyl, halogenated C₁-C₁₂ alkyl, C₁-C₁₂ alkoxy, halogenated C₁-C₁₂ alkoxy, C₃-C₁₂ cycloalkyl, aryl or heteroaryl substituted by one to three R₆, or biphenyl- or phenoxyphenyl substituted by one or more R₆;
R₅ is selected from hydroxyl, carbonyl, methoxy, methoxyoximido, formyl, halogen, C₁-C₁₂ alkyl, halogenated C₁-C₁₂ alkyl, C₁-C₁₂ alkoxy, or halogenated C₁-C₁₂ alkoxy; and
R₆ is selected from halogen, hydroxyl, amino, cyano, nitro, C₁-C₁₂ alkyl, halogenated C₁-C₁₂ alkyl, C₁-C₁₂ alkoxy, halogenated C₁-C₁₂ alkoxy, C₃-C₁₂ cycloalkyl, C₁-C₁₂ alkylthio, halogenated C₁-C₁₂ alkylthio, C₁-C₁₂ alkylamino, halogenated C₁-C₁₂ alkylamino, di(C₁-C₁₂ alkyl)amino, halogenated di(C₁-C₁₂ alkyl)amino, C₂-C₁₂ alkenyl, C₂-C₁₂ alkynyl, C₂-C₁₂ alkenoxy, halogenated C₂-C₁₂ alkenoxy, C₂-C₁₂ alkynyloxy, halogenated C₂-C₁₂ alkynyloxy, C₁-C₁₂ alkylsulfonyl, halogenated C₁-C₁₂ alkylsulfonyl, C₁-C₁₂ alkylcarbonyl, halogenated C₁-C₁₂ alkylcarbonyl, C₁-C₁₂ alkoxycarbonyl, halogenated C₁-C₁₂ alkoxycarbonyl, C₁-C₁₂ alkoxy C₁-C₁₂ alkyl, halogenated C₁-C₁₂ alkoxy C₁-C₁₂ alkyl, C₁-C₁₂ alkylthio C₁-C₁₂ alkyl, halogenated C₁-C₁₂ alkylthio C₁-C₁₂ alkyl, C₁-C₁₂ alkoxycarbonyl C₁-C₁₂ alkyl, halogenated C₁-C₁₂ alkoxycarbonyl C₁-C₁₂ alkyl, C₁-C₁₂ alkylthiocarbonyl C₁-C₁₂ alkyl, halogenated C₁-C₁₂ alkylthiocarbonyl C₁-C₁₂ alkyl, C₁-C₁₂ alkylcarbonyloxy, halogenated C₁-C₁₂ alkylcarbonyloxy, C₁-C₁₂ alkoxycarbonyloxy, halogenated C₁-C₁₂ alkoxycarbonyloxy, C₁-C₁₂ alkylsulfonyloxy, halogenated C₁-C₁₂ alkylsulfonyloxy, C₁-C₁₂ alkoxy C₁-C₁₂ alkoxy or halogenated C₁-C₁₂ alkoxy C₁-C₁₂ alkoxy.

2. The butenolide compound containing a thiazolidinone structure of claim 1, wherein, in the compounds represented by the general formula I:
R₁ and R₂ are each independently selected from hydrogen or C₁-C₄ alkyl; or, R₁ and R₂, together with the carbon atom bonded therewith, can form C₅-C₆ cycloalkyl or C₅-C₆ cyclic heteroalkyl, the heteroatom in the heteroalkyl being N, O, or S, wherein the hydrogen in the C₃-C₁₂ cycloalkyl or the C₃-C₁₂ cyclic heteroalkyl can be monosubstituted by R₅;
R₃ is selected from hydrogen, C₁-C₄ alkyl, C₁-C₄ cycloalkyl, unsubstituted aryl or heteroaryl, or aryl or heteroaryl containing one to three R₆ substituents;
R₄ is selected from aryl or heteroaryl substituted by one to three R₆, biphenyl- or phenoxyphenyl substituted by one or two R₆;
R₅ is selected from hydroxyl, carbonyl, methoxy, methoxyoximido, formyl, halogen, C₁-C₄ alkyl, and halogenated C₁-C₄ alkyl; and
R₆ is selected from halogen, hydroxyl, amino, cyano, nitro, C₁-C₄ alkyl, halogenated C₁-C₄ alkyl, C₁-C₄ alkoxy, halogenated C₁-C₄ alkoxy, C₃-C₄ cycloalkyl, C₁-C₄ alkylthio, halogenated C₁-C₄ alkylthio, C₁-C₄ alkylamino, halogenated C₁-C₄ alkylamino, C₁-C₄ alkylsulfonyl, halogenated C₁-C₄ alkylsulfonyl, C₁-C₄ alkylcarbonyl, and halogenated C₁-C₄ alkylcarbonyl.

3. The butenolide compound containing a thiazolidinone structure of claim 1, wherein, in the compounds represented by the general formula I:
R₁ and R₂ are each independently selected from hydrogen, methyl, ethyl, n-propyl, isopropyl, n-butyl, sec-butyl, isobutyl, tert-butyl, cyclopropyl, or cyclobutyl; or, R₁ and R₂, together with the carbon atom bonded therewith, can form a five-membered or six-membered carbon ring, and the hydrogen on the carbon ring can be monosubstituted by R₅;
R₃ is selected from hydrogen, methyl, ethyl, n-propyl, isopropyl, n-butyl, sec-butyl, isobutyl, tert-butyl, cyclopropyl, cyclobutyl, unsubstituted aryl or heteroaryl, or aryl or heteroaryl substituted by one to three R₆;
R₄ is selected from aryl or heteroaryl substituted by one to three R₆, biphenyl- or phenoxyphenyl substituted by one or two R₆;
R₅ is selected from hydroxyl, carbonyl, methoxy, methoxyoximido, or halogen; and
R₆ is selected from halogen, hydroxyl, amino, cyano, nitro, C₁-C₄ alkyl, halogenated C₁-C₄ alkyl, C₁-C₄ alkoxy, halogenated C₁-C₄ alkoxy, C₁-C₄ alkylthio or halogenated C₁-C₄ alkylthio.

4. The butenolide compound containing a thiazolidinone structure of claim 1, wherein, in the compounds represented by the general formula I:
R₁ and R₂ are methyl respectively;
R₁ and R₂, together with the carbon atom bonded therewith, can form a saturated five-membered or six-membered carbon ring, and the hydrogen on the carbon ring can be monosubstituted by R₅;
R₃ is selected from methyl or phenyl substituted by R₆;
R₄ is selected from phenyl substituted by R₆, biphenyl- or phenoxyphenyl substituted by one or two R₆;
R₅ is selected from methoxy or methoxyoximido; and
R₆ is selected from methyl, tert-butyl, halogen, nitro, methylthio, methoxy, or trifluoromethyl.

5. An intermediate of butenolide compounds containing a thiazolidinone structure, wherein the intermediate is represented by the general formula 3: 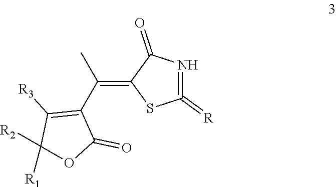
R is one selected from NH and S;
R₁ and R₂ are each independently selected from hydrogen or C₁-C₁₂ alkyl; or, R₁ and R₂, together with the carbon atom bonded therewith, form C₃-C₁₂ cycloalkyl or C₃-C₁₂ cyclic heteroalkyl, the heteroatom in the heteroalkyl being N, O, or S, wherein the hydrogen in the C₃-C₁₂ cycloalkyl or the C₃-C₁₂ cyclic heteroalkyl may be monosubstituted or polysubstituted by R₅;
R₃ is selected from hydrogen, hydroxyl, C₁-C₁₂ alkyl, C₅-C₇ cycloalkyl, unsubstituted aryl or heteroaryl, or aryl or heteroaryl containing 1-3 R₆ substituents;
R₄ is selected from phenyl substituted by R₆, biphenyl- or phenoxyphenyl substituted by one or two R₆;
R₅ is selected from hydroxyl, carbonyl, methoxy, methoxyoximido, formyl, halogen, C₁-C₁₂ alkyl, halogenated C₁-C₁₂ alkyl, C₁-C₁₂ alkoxy, or halogenated C₁-C₁₂ alkoxy; and
R₆ is selected from halogen, hydroxyl, amino, cyano, nitro, C₁-C₁₂ alkyl, halogenated C₁-C₁₂ alkyl, C₁-C₁₂ alkoxy, halogenated C₁-C₁₂ alkoxy, C₃-C₁₂ cycloalkyl, C₁-C₁₂ alkylthio, halogenated C₁-C₁₂ alkylthio, C₁-C₁₂ alkylamino, halogenated C₁-C₁₂ alkylamino, di(C₁-C₁₂ alkyl)amino, halogenated di(C₁-C₁₂ alkyl)amino, C₂-C₁₂ alkenyl, C₂-C₁₂ alkynyl, C₂-C₁₂ alkenoxy, halogenated C₂-C₁₂ alkenoxy, C₂-C₁₂ alkynyloxy, halogenated C₂-C₁₂ alkynyloxy, C₁-C₁₂ alkylsulfonyl, halogenated C₁-C₁₂ alkylsulfonyl, C₁-C₁₂ alkylcarbonyl, halogenated C₁-C₁₂ alkylcarbonyl, C₁-C₁₂ alkoxycarbonyl, halogenated C₁-C₁₂ alkoxycarbonyl, C₁-C₁₂ alkoxy C₁-C₁₂ alkyl, halogenated C₁-C₁₂ alkoxy C₁-C₁₂ alkyl, C₁-C₁₂ alkylthio C₁-C₁₂ alkyl, halogenated C₁-C₁₂ alkylthio C₁-C₁₂ alkyl, C₁-C₁₂ alkoxycarbonyl C₁-C₁₂ alkyl, halogenated C₁-C₁₂ alkoxycarbonyl C₁-C₁₂ alkyl, C₁-C₁₂ alkylthiocarbonyl C₁-C₁₂ alkyl, halogenated C₁-C₁₂ alkylthiocarbonyl C₁-C₁₂ alkyl, C₁-C₁₂ alkylcarbonyloxy, halogenated C₁-C₁₂ alkylcarbonyloxy, C₁-C₁₂ alkoxycarbonyloxy, halogenated C₁-C₁₂ alkoxycarbonyloxy, C₁-C₁₂ alkylsulfonyloxy, halogenated C₁-C₁₂ alkylsulfonyloxy, C₁-C₁₂ alkoxy C₁-C₁₂ alkoxy or halogenated C₁-C₁₂ alkoxy C₁-C₁₂ alkoxy.

6. A preparation method of the butenolide compound containing a thiazolidinone structure claim 1, wherein the compound represented by the general formula I is synthesized according to the following scheme: 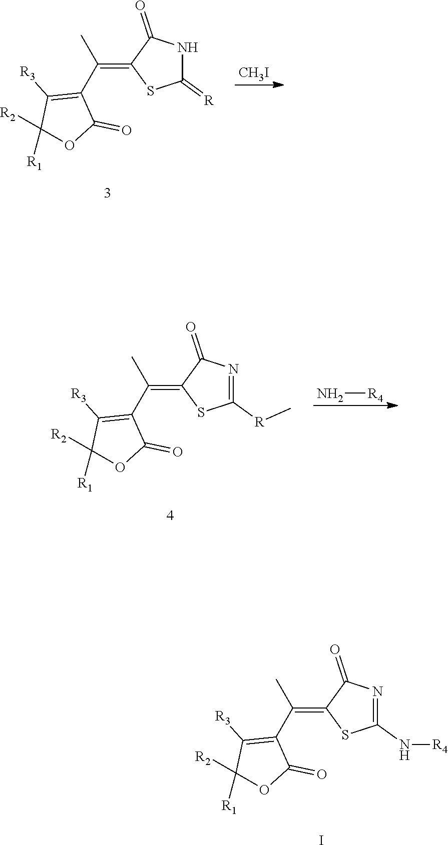
the definitions of R, R₁, R₂, R₃, and R₄ in the formula are the same as those described in claim 1.

7. The preparation method of claim 6, characterized by comprising the following steps:
(1) in a first solvent, subjecting the intermediate compound represented by the general formula 3 to methylation reaction under the action of a base to obtain a compound 4, the base being one or more selected from potassium carbonate, cesium carbonate, sodium hydride, sodium ethoxide, and sodium hydroxide, the first solvent being selected from acetonitrile and/or acetone, and the reaction temperature being raised from room temperature to a reflux temperature; and
(2) in a second solvent, reacting the compound 4 with NH₂—R₄ under reflux in presence of an acid catalyst to obtain the compound represented by the general formula I, the second solvent being selected from toluene and/or xylene, and the acid being one or more selected from oxalic acid, acetic acid, and propionic acid.

8. A preparation method of the intermediate of butenolide compounds containing a thiazolidinone structure of claim 5, wherein synthesis scheme is as follows: 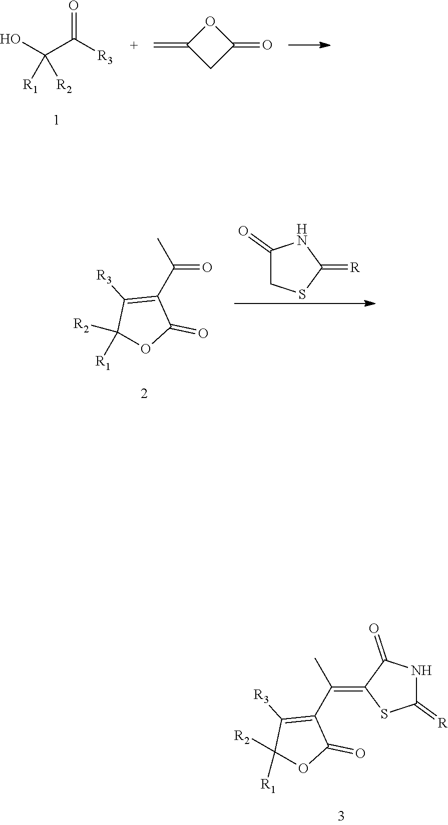
wherein the definitions of R, R₁, R₂, and R₃ in the formula are the same as those described in claim 1.

9. The preparation method of claim 8, characterized by comprising:
1) reacting compound 1 with diketene in the presence of a catalyst to obtain 3-acetylbutenolide compound 2, the catalyst being selected from triethylamine and/or diisopropylethylamine; and
2) in a third solvent, reacting the compound 2 with 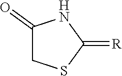
under the action of an organic base under reflux to obtain the intermediate compound represented by the general formula 3, wherein R is one selected from NH and S; the organic base is one or more selected from ethanolamine, piperidine, pyridine, sodium acetate, and ammonium acetate, and the third solvent is selected from toluene and/or xylene.

10. (canceled)

11. The butenolide compound containing a thiazolidinone structure of claim 2, wherein, in the compounds represented by the general formula I:
R₁ and R₂ are each independently selected from hydrogen, methyl, ethyl, n-propyl, isopropyl, n-butyl, sec-butyl, isobutyl, tert-butyl, cyclopropyl, or cyclobutyl; or, R₁ and R₂, together with the carbon atom bonded therewith, can form a five-membered or six-membered carbon ring, and the hydrogen on the carbon ring can be monosubstituted by R₅;
R₃ is selected from hydrogen, methyl, ethyl, n-propyl, isopropyl, n-butyl, sec-butyl, isobutyl, tert-butyl, cyclopropyl, cyclobutyl, unsubstituted aryl or heteroaryl, or aryl or heteroaryl substituted by one to three R₆;
R₄ is selected from aryl or heteroaryl substituted by one to three R₆, biphenyl- or phenoxyphenyl substituted by one or two R₆;
R₅ is selected from hydroxyl, carbonyl, methoxy, methoxyoximido, or halogen; and
R₆ is selected from halogen, hydroxyl, amino, cyano, nitro, C₁-C₄ alkyl, halogenated C₁-C₄ alkyl, C₁-C₄ alkoxy, halogenated C₁-C₄ alkoxy, C₁-C₄ alkylthio or halogenated C₁-C₄ alkylthio.

12. The butenolide compound containing a thiazolidinone structure of claim 2, wherein, in the compounds represented by the general formula I:
R₁ and R₂ are methyl respectively;
R₁ and R₂, together with the carbon atom bonded therewith, can form a saturated five-membered or six-membered carbon ring, and the hydrogen on the carbon ring can be monosubstituted by R₅;
R₃ is selected from methyl or phenyl substituted by R₆;
R₄ is selected from phenyl substituted by R₆, biphenyl- or phenoxyphenyl substituted by one or two R₆;
R₅ is selected from methoxy or methoxyoximido; and
R₆ is selected from methyl, tert-butyl, halogen, nitro, methylthio, methoxy, or trifluoromethyl.

13. The butenolide compound containing a thiazolidinone structure of claim 3,
wherein, in the compounds represented by the general formula I:
R₁ and R₂ are methyl respectively;
R₁ and R₂, together with the carbon atom bonded therewith, can form a saturated five-membered or six-membered carbon ring, and the hydrogen on the carbon ring can be monosubstituted by R₅;
R₃ is selected from methyl or phenyl substituted by R₆;
R₄ is selected from phenyl substituted by R₆, biphenyl- or phenoxyphenyl substituted by one or two R₆;
R₅ is selected from methoxy or methoxyoximido; and
R₆ is selected from methyl, tert-butyl, halogen, nitro, methylthio, methoxy, or trifluoromethyl.
